SEQUENCE LISTING (26 sequences)

SEQ ID NO: 1 - protein, 826 aa, Homo sapiens
  hypoxia induced factor 1alpha (HIF-lalpha) protein isoform 1
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 2 - protein, 735 aa, Homo sapiens
  hypoxia induced factor 1alpha (HIF-lalpha) protein isoform 2
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGII

SEQ ID NO: 3 - protein, 836 aa, Mus musculus
  hypoxia induced factor 1alpha (HIF-lalpha) protein
MEGAGGENEKKKMSSERRKEKSRDAARSRRSKESEVFYELAHQLPLPHNVSSHLDKASVMRLTISYLRVRKLLDAGGLDSEDEMKAQMDCFYLKALDGFVMVLTDDGDMVYISDNVNKYMGLTQFELTGHSVFDFTHPCDHEEMREMLTHRNGPVRKGKELNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPSSETPKPLRSSADPALNQEVALKLESSPESLGLSFTMPQIQDQPASPSDGSTRQSSPERLLQENVNTPNFSQPNSPSEYCFDVDSDMVNVFKLELVEKLFAEDTEAKNPFSTQDTDLDLEMLAPYIPMDDDFQLRSFDQLSPLESNSPSPPSMSTVTGFQQTQLQKPTITATATTTATTDESKTETKDNKEDIKILIASPSSTQVPQETTTAKASAYSGTHSRTASPDRAGKRVIEQTDKAHPRSLKLSATLNQRNTVPEEELNPKTIASQNAQRKRKMEHDGSLFQAAGIGTLLQQPGDCAPTMSLSWKRVKGFISSEQNGTEQKTIILIPSDLACRLLGQSMDESGLPQLTSYDCEVNAPIQGSRNLLQGEELLRALDQVN

SEQ ID NO: 4 - protein, 823 aa, Rattus norvegicus
  hypoxia induced factor 1alpha (HIF-lalpha) protein
MEGAGGENEKKNRMSSERRKEKSRDAARSRRSKESEVFYELAHQLPLPHNVSSHLDKASVMRLTISYLRVRKLLGAGDLDIEDEMKAQMNCFYLKALDGFVMVLTDDGDMIYISDNVNKYMGLTQFELTGHSVFDFTHPCDHEEMREMLTHRNGPVRKGKEQNTQRSFFLRMKCTLTSRGRTMNIKSATWKVLHCTGHIHVYDTSSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTESVLKPVESSDMKMTQLFTKVESEDTSCLFDKLKKEPDALTLLAPAAGDTIISLDFGSDDTETEDQQLEDVPLYNDVMFPSSNEKLNINLAMSPLPASETPKPLRSSADPALNQEVALKLESSPESLGLSFTMPQIQDQPASPSDGSTRQSSPEPNSPSEYCFDVDSDMVNVFKLELVEKLFAEDTEAKNPFSAQDTDLDLEMLAPYIPMDDDFQLRSFDQLSPLESNSPSPPSVSTVTGFQQTQLQKPTITVTATATATTDESKAVTKDNIEDIKILIASPPSTQVPQEMTTAKASAYSGTHSRTASPDRAGKRVIEKTDKAHPRSLNLSVTLNQRNTVPEEELNPKTIALQNAQRKRKMEHDGSLFQAAGIGTLLQQPGDRAPTMSLSWKRVKGYISSEQDGMEQKTIFLIPSDLACRLLGQSMDESGLPQLTSYDCEVNAPIQGSRNLLQGEELLRALDQVN

SEQ ID NO: 5 - DNA, 3958 bp, Homo sapiens
  hypoxia induced factor 1alpha (HIF-lalpha) cDNA variant 1
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggaaaaaaaaaaaaaaaaaaa

SEQ ID NO: 6 - DNA, 3812 bp, Homo sapiens
  hypoxia induced factor 1alpha (HIF-lalpha) cDNA variant 2
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccagg

SEQ ID NO: 7 - DNA, 3973 bp, Mus musculus
  hypoxia induced factor 1alpha (HIF-lalpha) cDNA
cgcgaggactgtcctcgccgccgtcgcgggcagtgtctagccaggccttgacaagctagccggaggagcgcctaggaacccgagccggagctcagcgagcgcagcctgcacgcccgcctcgcgtcccgggggggtcccgcctcccaccccgcctctggacttgtctctttccccgcgcgcgcggacagagccggcgtttaggcccgagcgagcccgggggccgccggccgggaagacaacgcgggcaccgattcgccatggagggcgccggcggcgagaacgagaagaaaaagatgagttctgaacgtcgaaaagaaaagtctagagatgcagcaagatctcggcgaagcaaagagtctgaagttttttatgagcttgctcatcagttgccacttccccacaatgtgagctcacatcttgataaagcttctgttatgaggctcaccatcagttatttacgtgtgagaaaacttctggatgccggtggtctagacagtgaagatgagatgaaggcacagatggactgtttttatctgaaagccctagatggctttgtgatggtgctaacagatgacggcgacatggtttacatttctgataacgtgaacaaatacatggggttaactcagtttgaactaactggacacagtgtgtttgattttactcatccatgtgaccatgaggaaatgagagaaatgcttacacacagaaatggcccagtgagaaaagggaaagaactaaacacacagcggagcttttttctcagaatgaagtgcaccctaacaagccgggggaggacgatgaacatcaagtcagcaacgtggaaggtgcttcactgcacgggccatattcatgtctatgataccaacagtaaccaacctcagtgtgggtacaagaaaccacccatgacgtgcttggtgctgatttgtgaacccattcctcatccgtcaaatattgaaattcctttagatagcaagacatttctcagtcgacacagcctcgatatgaaattttcttactgtgatgaaagaattactgagttgatgggttatgagccggaagaacttttgggccgctcaatttatgaatattatcatgctttggattctgatcatctgaccaaaactcaccatgatatgtttactaaaggacaagtcaccacaggacagtacaggatgcttgccaaaagaggtggatatgtctgggttgaaactcaagcaactgtcatatataatacgaagaactcccagccacagtgcattgtgtgtgtgaattatgttgtaagtggtattattcagcacgacttgattttctcccttcaacaaacagaatctgtgctcaaaccagttgaatcttcagatatgaagatgactcagctgttcaccaaagttgaatcagaggatacaagctgcctttttgataagcttaagaaggagcctgatgctctcactctgctggctccagctgccggcgacaccatcatctctctggattttggcagcgatgacacagaaactgaagatcaacaacttgaagatgttccattatataatgatgtaatgtttccctcttctaatgaaaaattaaatataaacctggcaatgtctcctttaccttcatcggaaactccaaagccacttcgaagtagtgctgatcctgcactgaatcaagaggttgcattaaaattagaatcaagtccagagtcactgggactttcttttaccatgccccagattcaagatcagccagcaagtccttctgatggaagcactagacaaagttcacctgagagacttcttcaggaaaacgtaaacactcctaacttttcccagcctaacagtcccagtgaatattgctttgatgtggatagcgatatggtcaatgtattcaagttggaactggtggaaaaactgtttgctgaagacacagaggcaaagaatccattttcaactcaggacactgatttagatttggagatgctggctccctatatcccaatggatgatgatttccagttacgttcctttgatcagttgtcaccattagagagcaattctccaagccctccaagtatgagcacagttactgggttccagcagacccagttacagaaacctaccatcactgccactgccaccacaactgccaccactgatgaatcaaaaacagagacgaaggacaataaagaagatattaaaatactgattgcatctccatcttctacccaagtacctcaagaaacgaccactgctaaggcatcagcatacagtggcactcacagtcggacagcctcaccagacagagcaggaaagagagtcatagaacagacagacaaagctcatccaaggagccttaagctgtctgccactttgaatcaaagaaatactgttcctgaggaagaattaaacccaaagacaatagcttcgcagaatgctcagaggaagcgaaaaatggaacatgatggctccctttttcaagcagcaggaattggaacattattgcagcaaccaggtgactgtgcacctactatgtcactttcctggaaacgagtgaaaggattcatatctagtgaacagaatggaacggagcaaaagactattattttaataccctccgatttagcatgcagactgctggggcagtcaatggatgagagtggattaccacagctgaccagttacgattgtgaagttaatgctcccatacaaggcagcagaaacctactgcagggtgaagaattactcagagctttggatcaagttaactgagcgtttcctaatctcattccttttgattgttaatgtttttgttcagttgttgttgtttgttgggtttttgtttctgttggttatttttggacactggtggctcagcagtctatttatattttctatatctaattttagaagcctggctacaatactgcacaaactcagatagtttagttttcatcccctttctacttaattttcattaatgctctttttaatatgttcttttaatgccagatcacagcacattcacagctcctcagcatttcaccattgcattgctgtagtgtcatttaaaatgcacctttttatttatttatttttggtgagggagtttgtcccttattgaattatttttaatgaaatgccaatataattttttaagaaagcagtaaattctcatcatgatcataggcagttgaaaactttttactcatttttttcatgttttacatgaaaataatgctttgtcagcagtacatggtagccacaattgcacaatatattttctttaaaaaaccagcagttactcatgcaatatattctgcatttataaaactagtttttaagaaattttttttggcctatggaattgttaagcctggatcatgaagcgttgatcttataatgattcttaaactgtatggtttctttatatgggtaaagccatttacatgatataaagaaatatgcttatatctggaaggtatgtggcatttatttggataaaattctcaattcagagaagttatctggtgtttcttgactttaccaactcaaaacagtccctctgtagttgtggaagcttatgctaatattgtgtaattgattatgaaacataaatgttctgcccaccctgttggtataaagacattttgagcatactgtaaacaaacaaacaaaaaatcatgctttgttagtaaaattgcctagtatgttgatttgttgaaaatatgatgtttggttttatgcactttgtcgctattaacatccttttttcatatagatttcaataagtgagtaattttagaagcattattttaggaatatagagttgtcatagtaaacatcttgttttttctatgtacactgtataaatttttcgttcccttgctctttgtggttgggtctaacactaactgtactgttttgttatatcaaataaacatcttctgtggaccaggaaaaaaaaaaaaaaaaaa

SEQ ID NO: 8 - DNA, 3718 bp, Rattus norvegicus
  hypoxia induced factor 1alpha (HIF-lalpha) cDNA
gacaccgcgggcaccgattcgccatggagggcgccggcggcgagaacgagaagaaaaataggatgagttccgaacgtcgaaaagaaaagtctagggatgcagcacgatctcggcgaagcaaagagtctgaagttttttatgagcttgctcatcagttgccacttccccacaacgtgagctcccatcttgataaagcttctgttatgaggctcaccatcagttacttacgtgtgaggaaacttctaggtgctggtgatcttgacattgaagatgaaatgaaagcacagatgaactgcttttatctgaaagccctggatggctttgttatggtgctaacagatgatggtgacatgatttacatttctgataacgtgaacaaatacatggggttgactcagtttgaactaactggacacagtgtgtttgattttacccatccatgtgaccatgaggaaatgagagaaatgcttacacacagaaatggcccagtgagaaaggggaaagaacaaaacacgcagcgaagcttttttctcagaatgaaatgtaccctaacaagccgggggaggacgatgaacatcaagtcagcaacgtggaaggtgctgcactgcacaggccacattcatgtgtatgataccagcagtaaccagccgcagtgtggctacaagaaaccgcctatgacgtgcttggtgctgatttgtgaacccattcctcatccatcaaacattgaaattcctttagacagcaagacatttctcagtcgacacagcctcgatatgaaattttcttactgtgatgaaaggattactgagttgatgggttatgagccagaagaacttttgggccgttcaatttatgaatattatcatgctttggactctgatcatctgaccaaaactcatcatgacatgtttactaaaggacaagtcaccacaggacagtacaggatgcttgcaaaaagaggtggatatgtctgggttgagactcaagcaactgttatatataatacgaagaactctcagccacagtgcattgtgtgtgtgaattatgttgtaagtggtattattcagcacgacttgattttctcccttcaacaaacagaatctgtcctcaaaccagttgaatcttcagatatgaaaatgacccagctgttcactaaagtggaatctgaggacacgagctgcctcttcgacaagcttaagaaagagcccgatgccctgactctgctagctccagcggctggggacacgatcatatcactggacttcggcagcgatgacacggaaactgaagaccaacaacttgaagatgtcccgttgtacaatgatgtaatgttcccctcttctaatgagaaattaaatataaatctggcaatgtctccattacctgcctctgaaactccaaagccacttcgaagtagtgctgatcctgcactgaatcaagaggttgcattgaagttagagtcaagcccagagtcactgggactttcttttaccatgccccagattcaagatcagccagcaagtccttctgatggaagcactagacaaagctcacctgagcctaacagtcccagtgagtactgctttgatgtggacagcgatatggtcaatgtattcaagttggaactggtggaaaaactgtttgctgaagacacagaagcgaagaatccattttcagctcaggacactgatttagacttggaaatgctggctccctatatcccaatggatgatgatttccagttacgttcctttgatcagttgtcaccattagagagcaattctccaagccctccgagtgtgagcacagttacaggattccagcagacccagttacagaaacctaccatcactgtcactgccaccgcaactgccaccactgatgaatcaaaagcagtgacgaaggacaatatagaagacattaaaatactgattgcatctccaccttctacccaagtacctcaagaaatgaccactgctaaggcatcagcatacagtggtactcacagtcggacagcctcaccagacagagcaggaaagagagtcatagaaaaaacagacaaagctcatccaaggagccttaacctatctgtcactttgaatcaaagaaatactgttcctgaagaagaattaaacccaaagacaatagctttgcagaatgctcagaggaagcgaaaaatggaacatgatggctccctttttcaagcagcaggaattggaacgttactgcagcaaccaggtgaccgtgcccctactatgtcgctttcttggaaacgagtgaaaggatacatatctagtgaacaggatggaatggagcagaagacaatttttttaataccctctgatttagcatgtagactgctggggcagtcaatggatgagagtggattaccacagctgaccagttacgattgtgaagttaatgctcccatacaaggcagcagaaacctactgcagggtgaagaattactcagagctttggatcaagttaactgagcttttcctaatctcattcctttgattgttaatttttgtgttcagttgttgttgttgtctgtggggtttcgtttctgttggttgttttggacactggtggctcagcagtctatttatattttctatatctcatttagaggcctggctacagtactgcaccaactcagatagtttagtttgggccccttcctccttcattttcactgatgctctttttaccatgtccttcgaatgccagatcacagcacattcacagctccccagcatttcaccaatgcattgctgtagtgtcgtttaaaatgcacctttttatttatttatttttggtgagggagtttgtcccttattgaattatttttaatgaaatgccaatataattttttaagaaggcagtaaatcttcatcatgatgataggcagttgaaaattttttactcatttttttcatgttttacatgaaaataatgctttgccagcagtacatggtagccacaattgcacaatatattttcttaaaaataccagcagttactcatgcatatattctgcatttataaaactagtttttaagaagaaactttttttggcctatggaattgttaagcctggatcatgatgctgttgatcttataatgattcttaaactgtatggtttctttatatgggtaaagccatttacatgatatagagagatatgcttatatctggaaggtatatggcatttatttggataaaattctcaattgagaagttatctggtgtttctttactttaccggctcaaaagaaaacagtccctatgtagttgtggaagcttatgctaatattgtgtaattgatattaaacattaaatgttctgcctatcctgttggtataaagacattttgagcatactgtaaacaaaaaaatcatgcattgttagtaaaattgcctagtatgttaatttgttgaaaatacgatgtttggttttatgcactttgtcgctattaacatcctttttttcatatagatttcaataattgagtaattttagaagcattattttagaaatatagagttgtcatagtaaacatcttgtttttttttctttttttctatgtacattgtataaatttttcattcccttgctctttgtagttgggtctaacactaactgtactgttttgttatatcaaataaacatcttctgtggaccaggaaaaaaaaaaaaaaaaaaaaaaaaaaa

SEQ ID NO: 9 - protein, 213 aa, Homo sapiens
  von Hippel-Lindau (VHL) protein isoform 1
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVQKDLERLTQERIAHQRMGD

SEQ ID NO: 10 - protein, 172 aa, Homo sapiens
  von Hippel-Lindau (VHL) protein isoform 2
MPRRAENWDEAEVGAEEAGVEEYGPEEDGGEESGAEESGPEESGPEELGAEEEMEAGRPRPVLRSVNSREPSQVIFCNRSPRVVLPVWLNFDGEPQPYPTLPPGTGRRIHSYRVYTLKERCLQVVRSLVKPENYRRLDIVRSLYEDLEDHPNVQKDLERLTQERIAHQRMGD

SEQ ID NO: 11 - protein, 181 aa, Mus musculus
  von Hippel-Lindau (VHL) protein
MPRKAASPEEAAGEPGPEEMEAGRPRPVLRSVNSREPSQVIFCNRSPRVVLPLWLNFDGEPQPYPILPPGTGRRIHSYRGHLWLFRDAGTHDGLLVNQTELFVPSLNVDGQPIFANITLPVYTLKERCLQVVRSLVKPENYRRLDIVRSLYEDLEDYPSVRKDIQRLSQEHLESQHLEEEP

SEQ ID NO: 12 - protein, 185 aa, Rattus norvegicus
  von Hippel-Lindau (VHL) protein
MPRKAASPEEAERMPGSEEIEAGRPRPVLRSVNSREPSQVIFCNRSPRVVLPLWLNFDGEPQPYPTLPPGTGRRIHSYRGHLWLFRDAGTHDGLLVNQTELFVPSLNVDGQPIFANITLPVYTLKERCLQVVRSLVKPENYRRLDIVRSLYEDLEDHPNVRKDIQRLTQEHLENQALGEEPEGVH

SEQ ID NO: 13 - DNA, 2968 bp, Homo sapiens
  von Hippel-Lindau (VHL) cDNA variant 1
cctcgcctccgttacaacggcctacggtgctggaggatccttctgcgcacgcgcacagcctccggccggctatttccgcgagcgcgttccatcctctaccgagcgcgcgcgaagactacggaggtcgactcgggagcgcgcacgcagctccgccccgcgtccgacccgcggatcccgcggcgtccggcccgggtggtctggatcgcggagggaatgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgaggtcacctttggctcttcagagatgcagggacacacgatgggcttctggttaaccaaactgaattatttgtgccatctctcaatgttgacggacagcctatttttgccaatatcacactgccagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattgaagatttctgttgaaacttacactgtttcatctcagcttttgatggtactgatgagtcttgatctagatacaggactggttccttccttagtttcaaagtgtctcattctcagagtaaaataggcaccattgcttaaaagaaagttaactgacttcactaggcattgtgatgtttaggggcaaacatcacaaaatgtaatttaatgcctgcccattagagaagtatttatcaggagaaggtggtggcatttttgcttcctagtaagtcaggacagcttgtatgtaaggaggtttgtataagtaattcagtgggaattgcagcatatcgtttaattttaagaaggcattggcatctgcttttaatggatgtataatacatccattctacatccgtagcggttggtgacttgtctgcctcctgctttgggaagactgaggcatccgtgaggcagggacaagtctttctcctctttgagaccccagtgcctgcacatcatgagccttcagtcagggtttgtcagaggaacaaaccaggggacactttgttagaaagtgcttagaggttctgcctctatttttgttggggggtgggagaggggaccttaaaatgtgtacagtgaacaaatgtcttaaagggaatcatttttgtaggaagcattttttataattttctaagtcgtgcactttctcggtccactcttgttgaagtgctgttttattactgtttctaaactaggattgacattctacagttgtgataatagcatttttgtaacttgccatccgcacagaaaatacgagaaaatctgcatgtttgattatagtattaatggacaaataagtttttgctaaatgtgagtatttctgttcctttttgtaaatatgtgacattcctgattgatttgggtttttttgttgttgttgttttgttttgttttgtttttttgagatggagtctcactcttgtcacccaggctggagtgcagtggcgccatctcggctcactgcaacctctgcctcctgggttcacgtaatcctcctgagtagctgggattacaggcgcctgccaccacgctggccaatttttgtacttttagtagagacagtgtttcgccatgttggccaggctggtttcaaactcctgacctcaggtgatccgcccacctcagcctcccaaaatggtgggattacaggtgtgtgggccaccgtgcctggctgattcagcattttttatcaggcaggaccaggtggcacttccacctccagcctctggtcctaccaatggattcatggagtagcctggactgtttcatagttttctaaatgtacaaattcttataggctagacttagattcattaactcaaattcaatgcttctatcagactcagttttttgtaactaatagatttttttttccacttttgttctactccttccctaatagctttttaaaaaaatctccccagtagagaaacatttggaaaagacagaaaactaaaaaggaagaaaaaagatccctattagatacacttcttaaatacaatcacattaacattttgagctatttccttccagcctttttagggcagattttggttggtttttacatagttgagattgtactgttcatacagttttataccctttttcatttaactttataacttaaatattgctctatgttagtataagcttttcacaaacattagtatagtctcccttttataattaatgtttgtgggtatttcttggcatgcatctttaattccttatcctagcctttgggcacaattcctgtgctcaaaaatgagagtgacggctggcatggtggctcccgcctgtaatcccagtactttgggaagccaaggtaagaggattgcttgagcccagaacttcaagatgagcctgggctcatagtgagaacccatctatacaaaaaatttttaaaaattagcatggcggcacacatctgtaatcctagctacttggcaggctgaggtgagaagatcattggagtttaggaattggaggctgcagtgagccatgagtatgccactgcactccagcctgggggacagagcaagaccctgcctcaaaaaaaaaaaaaaaaaa

SEQ ID NO: 14 - DNA, 2845 bp, Homo sapiens
  von Hippel-Lindau (VHL) cDNA variant 2
cctcgcctccgttacaacggcctacggtgctggaggatccttctgcgcacgcgcacagcctccggccggctatttccgcgagcgcgttccatcctctaccgagcgcgcgcgaagactacggaggtcgactcgggagcgcgcacgcagctccgccccgcgtccgacccgcggatcccgcggcgtccggcccgggtggtctggatcgcggagggaatgccccggagggcggagaactgggacgaggccgaggtaggcgcggaggaggcaggcgtcgaagagtacggccctgaagaagacggcggggaggagtcgggcgccgaggagtccggcccggaagagtccggcccggaggaactgggcgccgaggaggagatggaggccgggcggccgcggcccgtgctgcgctcggtgaactcgcgcgagccctcccaggtcatcttctgcaatcgcagtccgcgcgtcgtgctgcccgtatggctcaacttcgacggcgagccgcagccctacccaacgctgccgcctggcacgggccgccgcatccacagctaccgagtgtatactctgaaagagcgatgcctccaggttgtccggagcctagtcaagcctgagaattacaggagactggacatcgtcaggtcgctctacgaagatctggaagaccacccaaatgtgcagaaagacctggagcggctgacacaggagcgcattgcacatcaacggatgggagattgaagatttctgttgaaacttacactgtttcatctcagcttttgatggtactgatgagtcttgatctagatacaggactggttccttccttagtttcaaagtgtctcattctcagagtaaaataggcaccattgcttaaaagaaagttaactgacttcactaggcattgtgatgtttaggggcaaacatcacaaaatgtaatttaatgcctgcccattagagaagtatttatcaggagaaggtggtggcatttttgcttcctagtaagtcaggacagcttgtatgtaaggaggtttgtataagtaattcagtgggaattgcagcatatcgtttaattttaagaaggcattggcatctgcttttaatggatgtataatacatccattctacatccgtagcggttggtgacttgtctgcctcctgctttgggaagactgaggcatccgtgaggcagggacaagtctttctcctctttgagaccccagtgcctgcacatcatgagccttcagtcagggtttgtcagaggaacaaaccaggggacactttgttagaaagtgcttagaggttctgcctctatttttgttggggggtgggagaggggaccttaaaatgtgtacagtgaacaaatgtcttaaagggaatcatttttgtaggaagcattttttataattttctaagtcgtgcactttctcggtccactcttgttgaagtgctgttttattactgtttctaaactaggattgacattctacagttgtgataatagcatttttgtaacttgccatccgcacagaaaatacgagaaaatctgcatgtttgattatagtattaatggacaaataagtttttgctaaatgtgagtatttctgttcctttttgtaaatatgtgacattcctgattgatttgggtttttttgttgttgttgttttgttttgttttgtttttttgagatggagtctcactcttgtcacccaggctggagtgcagtggcgccatctcggctcactgcaacctctgcctcctgggttcacgtaatcctcctgagtagctgggattacaggcgcctgccaccacgctggccaatttttgtacttttagtagagacagtgtttcgccatgttggccaggctggtttcaaactcctgacctcaggtgatccgcccacctcagcctcccaaaatggtgggattacaggtgtgtgggccaccgtgcctggctgattcagcattttttatcaggcaggaccaggtggcacttccacctccagcctctggtcctaccaatggattcatggagtagcctggactgtttcatagttttctaaatgtacaaattcttataggctagacttagattcattaactcaaattcaatgcttctatcagactcagttttttgtaactaatagatttttttttccacttttgttctactccttccctaatagctttttaaaaaaatctccccagtagagaaacatttggaaaagacagaaaactaaaaaggaagaaaaaagatccctattagatacacttcttaaatacaatcacattaacattttgagctatttccttccagcctttttagggcagattttggttggtttttacatagttgagattgtactgttcatacagttttataccctttttcatttaactttataacttaaatattgctctatgttagtataagcttttcacaaacattagtatagtctcccttttataattaatgtttgtgggtatttcttggcatgcatctttaattccttatcctagcctttgggcacaattcctgtgctcaaaaatgagagtgacggctggcatggtggctcccgcctgtaatcccagtactttgggaagccaaggtaagaggattgcttgagcccagaacttcaagatgagcctgggctcatagtgagaacccatctatacaaaaaatttttaaaaattagcatggcggcacacatctgtaatcctagctacttggcaggctgaggtgagaagatcattggagtttaggaattggaggctgcagtgagccatgagtatgccactgcactccagcctgggggacagagcaagaccctgcctcaaaaaaaaaaaaaaaaaa

SEQ ID NO: 15 - DNA, 2730 bp, Mus musculus
  von Hippel-Lindau (VHL) cDNA
cgccttcaagttgcccggagcgcgtctgggaccggcagacctgtcccggagcgcgtagatccgcacgcacgcacgcgccgcccacgtccagcttgcgaatccgagggacccgttccaataatgccccggaaggcagccagtccagaggaggcggcgggggagcccggtcctgaggagatggaggctgggcggccgcggccggtgctgcgctcggtgaactcgcgcgagccctctcaggtcatcttctgcaaccgcagcccgcgcgtcgtgctgcctttgtggctcaacttcgacggcgagcctcagccctacccgatcttaccaccgggcaccggccgccgcatccacagctaccgaggtcatctttggctcttcagggatgcggggacccatgatggacttctggttaaccaaacggagctgtttgtgccatccctcaatgtcgatggacagcctatttttgccaacatcacattgccagtgtataccctgaaagagcggtgccttcaggttgtgcggagcctggtcaagcctgagaactacaggagactggacatcgtcaggtcactctatgaggatttggaggactacccaagtgtgcggaaggacatacagcgactgagccaagagcaccttgagagtcagcacctggaagaggagccttgaaggagtccatggagattaagtgttcctgagtttcagccttgatggtccgagattgatctacacataggacaggtcactttctttcagttttaaaatggttcattcttggagtaaaactatccatcacgtaaaagaaagttaactaacatccctgggctttgtagtgtttaagaataaacatgcaaagtgccactgcgtctgccctttgtagagcactcacccgagggaggaagacgttttcagttttgcttcctggtgaggctggaagttgagtgtaaggatgactgtgtaataagctcagcagcaggagttgtactgtgtcctttcatttgagcagagggctcttgcttgggaaggcagagaaggcccttcccaaacgctgggacagacctcctgtggaggcccgctgcctaaagcgtggagtcttcagtcatgaatgttggacgaagaaaccattggatgcttggtgggagagttgggagggcctggctctttgtctgaggagagccttaagtgttcatgtaaggaacagcttagcagcttctgatttcccacgtcccagcactttctctctgttttttattttatttttgagacttttgtatagcccaggccagcctcagtcactcagcagaggattcctgaccctctgggattatgtctcctgacattgggaacacatgtgtgcacacagcctgctctgctcttccttttcccatgttgagacgggctcctgctgtagtcctgcctggcctgagctggagtcggtcctgcctcctgaccgaccgtctggctgctttctaagctgggtagacttcccatactcactgttacagacagttacagcagagaagaaccatccttgtgtaagtctggttttgagagctcctgtctctgaggaagctgttggcaggagcagggatttctgttccttcttgtcagcccctagcattccactttgttttggcaggcgctggttctgctttgtaggtggaagtgaagctccactcagccacagactccagacagtgggtcacagagcagtctggaatgtgtagcagtcttatggatggtcacagcttttcttacatattcatattctatcaaacgttttgtaaagtataattttttatttatttttaatccccttagagaaaaagtaaaagtagagaagcctgggtattgtgtacacaacagaaaggacagtcaaggaaagggaagcagaaaggctcttatgtactctcagcttcatgcaacccctgtaactggccgcatatgtagggcatattttaattggttaaagcagtttgttgtggctgaggtcataagatatatacagttttgtatcctgatttttcactgaacttcattgttttttgtttgcttgcttttcccttcttctttgttttttgctttattgagatagggtttctctgtagcccaggctgtcctagaactccctttgtagaccaaacatctgattactggcctcacatggagagatctgtctctgcttttccagcgctgggattgaagtaggcctaatcttcatctaacttcttagcttgtcttttccatgttactacacacactgtgtaaataccagtgtgctaccctccttctttgagggtgactgttgggagtatttgtccaaatcctgggctccttgagtgcagactgatgctctaagatcagcaaaatgactgaactacatcctaacacttcccagtgtcctctcagtgtctcttcttatctgcagcattgcaggagtgaagtggccacagaccagtgtggaggcctgtgtgtgggaagtcagcaggtgccaagctattgaaacattggttttatttttttaaaccgatctctgtctctaaaagatgtgatgtttgtgttattcagctggaagtttgaatactgttctctgtgctgtccaacaagtgaagagttatgaaatgagccacttgtcctgggacatcctgtaatgagttcctcgggcttttctcctgtgagttttctgaaaaaagctgacctttttagtacaataaacggtgctaattgaaggaa

SEQ ID NO: 16 - DNA, 2807 bp, Rattus norvegicus
  von Hippel-Lindau (VHL) cDNA
ccgcgttaaagccggacggagtacttctattgctggcagacctgccccggagcgcgtaaacccgcacgctcgcgccgcccacgtccagctcgcgaatccggaggcgtccggttccaataatgccccggaaggcagctagtccagaggaggcagaaaggatgccgggctctgaagagatagaggctgggcggccgcggccggttttacgctctgtgaactcgcgcgaaccctctcaggtcatcttctgcaaccgcagcccgcgcgtcgtgctgcctttgtggctcaactttgatggtgagcctcagccctacccgaccttaccaccgggcaccggccgccgcatccacagctaccgaggtcacctttggctcttcagggatgcggggacccatgatggacttctggttaaccaaacggaactgtttgtgccatccctcaatgttgatggacagcctatttttgccaacatcacattgccagtgtataccctgaaagagcggtgccttcaggttgtacggagcctggtcaagcctgagaactacaggaggctggacatcgtcaggtcgctctatgaagacttggaagaccacccaaatgtgcggaaagacatacagcggctgacccaagagcacctcgagaatcaggccctgggagaggagcctgaaggagtccactgagattacttggtcctgaatttcggccttgatggtctgaaatcgatctacacataggacaggtcactttctttcggttttaaaatggttcattcttggagtaaaatatccatcatgtaaaagaaagttaactaacatcactgagctttgtagtgtttaagactaaacctgcaaagtgccaccgagtctgccctttctagagcattcatcagaagaagctgttttcagttttgcttcctagtaaggctggatgttcagcataaggatgttagtgtgagcgattcgcgaggagttgctctgtgtcctctaatctgaagaatacagcctcgtatgaacagagccccaccgctcacttggggaaggctgagaaggccctaatgctgggatggacatcctgtagaggcccagtgcctaatgtgtgtggagtcttcagtcagtgttggaggaacaaaccattgggtacttagtgggagagttggagggcctggctctctgtctgaggaaatcttaagtgtacacgtaaaggaacaacttagcagcttctgtttagaaagaactttctcttgagtttccatgtctcagcactttctttctactttgttagtttattttattttatttttgagacttgtgtagcccaggtcagcctcagtcactccggaggggatgctggtcttgaattccggatcctctgggagtgtgtcagcattgggagcacatgggtcgaccacagtctgctctgttcttgcttttcccattttgagacgggctcatactgtagccctgcctggcctcaagctagagtcagccctgtctccccacccagtctggctgcttagactgggtgggcttcccatattcactgccccagacagcagagacgagctgtccttgtgtttaggtctggttttgagagttccttctctgaggaagctgctggcaggagcagggtttctgttccttcccgtcagcccctagcattccagctgggcgccgcactctgctttggcaggcgctagttatgcttcgatgggagaggggagtgaagctccctcagccacacactccaaaccatgggtcccagagcagtctgcaatgagtagtagtttgtgggcagtcacagcttttcttacatattcaaatattgatgttcactacttctatcaaacattttgtagaacataattttttatttgtttttaattcctcagcagagaaaaatgagagaagcttgggcattgtgtgtggcacacaccagaaaggacagtcagctgagagggtgcagaaaggctcttgtgcgctggctctcagctgcatgtagtcctgtaacccagtgttctctctagtacacattttaattggttaaagcactttgtcctggccgagatcataaggtatatacagttttgtggcctgatttttcattgaacttcattgttttgttttcccctctttttcgtttttggttttgtttttggtttgttttttgctttttgttttatcaagatagagtttctctgtgtagcccaggctgtcctggaactccctttgtagaccaaacaactgattactggcctcaaacggagatctgtctgcctctgcctctccagtgctgggattgaagtatgcctaatcttcatctaacttagtaacttgtcttttccatgttactgcacacactgtctaaattccggtgtgctagcctctccctttgagggtgactgttgggagtatttgtctcagtcctgggctccttgagtgcagtctgatgctctaagatcagcaaaatcactgaacttaatactgacactcctgccgccctctcagcgtctctccttagtctgcagcattccgtgagtgaagcggcacaggccagtgtggagcctgtgtgtgggaagtcagcaggtgccgagctattgcaacattggttttattttttaagcctatctctgtctctaaaagatgtaatgtttgtcttatttaactggaagtttgaatactgttctctgtgccttaggacatgagtcacttgtccttgcacatcctgtaagtgtgttcctcagactttgtactctcctttgagttctctgaaaaagctgacctttagtacaataaacggtgctaagtgaagga

SEQ ID NO: 17 - RNA, 21 nt, Homo sapiens
  von Hippel-Lindau (VHL) siRNA
cggccuacggugcuggagguu

SEQ ID NO: 18 - RNA, 21 nt, Homo sapiens
  von Hippel-Lindau (VHL) siRNA
gacuacggaggucgacucguu

SEQ ID NO: 19 - RNA, 21 nt, Homo sapiens
  von Hippel-Lindau (VHL) siRNA
ugccccggagggcggagaauu

SEQ ID NO: 20 - RNA, 21 nt, Homo sapiens
  von Hippel-Lindau (VHL) siRNA
cugggacgaggccgagguauu

SEQ ID NO: 21 - protein, 16 aa, Artificial Sequence
  synthetic peptide; matched amino acid sequence for HIF-lalpha
SIYEYYHALDSDHLTK

SEQ ID NO: 22 - protein, 27 aa, Artificial Sequence
  synthetic peptide; matched amino acid sequence for HIF-lalpha
PPMXTCLVLICEPIPHPSNIEIPLDSK

SEQ ID NO: 23 - protein, 19 aa, Artificial Sequence
  synthetic peptide; matched amino acid sequence for HIF-lalpha
TFLSRHSLDMKXFSYCDER

SEQ ID NO: 24 - protein, 11 aa, Artificial Sequence
  synthetic peptide; matched amino acid sequence for HIF-lalpha
TMXNIKSATWK

SEQ ID NO: 25 - protein, 14 aa, Artificial Sequence
  synthetic peptide; matched amino acid sequence for HIF-2alpha
ENLTLKXNGSGFGK

SEQ ID NO: 26 - protein, 11 aa, Artificial Sequence
  synthetic peptide; matched amino acid sequence for HIF-2alpha
MXRSAKDFGAR

(19) United States
(12) Patent Application Publication
(10) Pub. No.: US 2013/0317084 A1
(43) Pub. Date: Nov. 28, 2013

(54) METHOD OF TREATING DIABETES

(71) Applicant: Garvan Institute of Medical Research C/-St. Vincent's Hospital, Darlinghurst (AU)

(72) Inventor: Jenny Gunton, Riverview (AU)

(73) Assignee: Garvan Institute of Medical Research C/-St. Vincent's Hospital, Darlinghurst (AU)

(21) Appl. No.: 13/957,922

(22) Filed: Aug. 2, 2013

Related U.S. Application Data
(62) Division of application No. 12/515,722, filed on Jan. 29, 2010, now Pat. No. 8,518,419.
(60) Provisional application No. 60/880,159, filed on Jan. 12, 2007.

(30) Foreign Application Priority Data
Nov. 21, 2006 (AU) 2006906499

(51) Int. Cl. A61K 31/713; A61K 31/164

(52) U.S. Cl. CPC A61K 31/713; A61K 31/164
USPC 514/44 A; 514/616

(57) ABSTRACT

The present invention relates to a method for treating a subject having or at risk of a diabetes-related disorder. In a preferred embodiment, the method involves increasing the level or activity of Hypoxia Induced Factor 1 (HIF-1α) in pancreatic-β-cells or insulin-sensitive tissues in the subject by administering to the subject an inhibitor of a protein that decreases the level or activity of HIF-1α. The present invention also relates to a method of transplanting pancreatic islet cells in a subject.

### FIELD OF THE INVENTION

The present invention provides a method for treating a subject having or at risk of a diabetes-related disorder. The present invention also provides a method of transplanting pancreatic islet cells in a subject.

### BACKGROUND OF THE INVENTION

Diabetes involves dysfunction of the pancreatic islet cells. In the case of type 1 diabetes, also referred to as insulin dependent diabetes mellitus (IDDM), dysfunction is initiated in the event of an immunological challenge. In the case of type 2 diabetes, also referred to as non-insulin dependent diabetes mellitus (NIDDM), islet dysfunction occurs upon exposure to a homeostatic challenge. Diabetes is associated with total β-cell mass, as well as the properties of individual β-cells.

Type 1 Diabetes and Insulitis.

Type 1 diabetes is a chronic autoimmune disease in which insulin-producing cells (β-cells) within the pancreatic islets of Langerhans are selectively targeted and destroyed by an infiltrate of immunological cells. This infiltrate causes an inflammatory affect on the islets, known as insulitis.

The development of type 1 diabetes is associated with an initial genetic susceptibility, although this susceptibility is insufficient for development of the disease. In susceptible individuals, it has been hypothesised that a triggering event leads to an active autoimmunity attack against β-cells, resulting in insulitis, islet β-cell dysfunction, diminished insulin secretion, and ultimately, β-cell destruction. β-cells comprise the majority of pancreatic islet cells. Overt type 1 diabetes onset characterised by hyperglycemia may not be diagnosed until years after an initial triggering event, at which point most of the pancreatic β-cells are destroyed. When overt diabetes is first recognised, some residual insulin production remains, as demonstrated by the presence of the connecting peptide (C peptide) of proinsulin in the serum. However, the individual usually requires injections of exogenous insulin. Complete β-cell destruction is determined when C peptide can no longer be detected in the circulation after stimulation with glucose or arginine.

The initiating factor(s) and specific sequence of events leading to type 1 diabetes, including the relative importance of different cell types and cytokines, are still widely debated. It is generally accepted that insulitis leading to type 1 diabetes involves cellular migration and infiltration of T lymphocytes, macrophages, and dendritic cells within the pancreatic islets. Immune stimulation of the newly infiltrated cells, and cytokine-regulated effects of such infiltration result in inflammation and β-cell destruction (Mandrup-Poulsen, 1996). Interleukin 1β (IL 1β), alone or in combination with tumor necrosis factor α (TNFα) and interferon γ (IFN γ), exhibits cytotoxicity toward n-cells in vitro (Cetkovic et al., 1994). This cytotoxicity is partly mediated through induction of free radicals such as nitric oxide (NO), the production of which is catalysed by inducible nitric oxide synthase (iNOS). NO released in n-cells leads to nuclear DNA fragmentation and apoptosis, a result which can be partially prevented by iNOS blockers. However, the blockers may not be used in vivo because of the various roles of NO in other organ systems.

Conventional treatment protocols for type 1 diabetes comprise regular administration, of insulin. Preferably, the insulin is administerered by injection. Other protocols have been suggested which include such immunomodulatory and immunosuppressive agents as levamisol, theophyllin, thymic hormones, ciamexone, antithymocyte globulin, interferon, cyclosporin, nicotinamide, gamma globulin infusion, plasmapheresis or white cell transfusion. Although these protocols may delay onset of type 1 diabetes, some undesirable side effects are observed. Treatment protocols after onset of type 1 diabetes are particularly problematic, since by the time diabetes is diagnosed in humans, insulitis has already progressed dramatically, resulting in a β-cell loss of more than 80%. Islet cell transplantation is a viable treatment for type 1 diabetes although graft rejection is still a major problem. Survival of transplanted islets requires effective immunosuppression, to block the immune response that leads to graft rejection. However, it is thought that the majority of islet death occurs in the first week post-transplant with up to 70% of β-cells also undergoing apoptotic cell death triggered by nonimmunological factors, such as hypoxia.

Type 2 Diabetes.

Type 2 diabetes often occurs in the face of normal, or even elevated levels of insulin. The condition appears to arise from β-cell dysfunction, usually combined with impaired ability of tissues to respond appropriately to insulin (i.e. insulin resistance), which challenges the homeostasis of blood glucose. Over time, many individuals with type 2 diabetes show decreased insulin production and require supplemental insulin to maintain blood glucose control, especially during times of stress or illness.

Conventional treatments for type 2 diabetes have not changed substantially in many years, and have significant limitations. While physical exercise and a reduction in caloric intake can improve the condition, compliance with such regimens is generally poor. Oral anti-diabetic drugs—the sulfonylureas, biguanides (metformin), thiazolidediones, α-glucosidase inhibitors (acarbose, miglitol), meglitinides (nateglinide, repaglinide) and exenatide can also be used to maintain blood glucose levels. Insulin therapy may also be used as an adjunct or alternative to oral medication therapy.

### SUMMARY OF THE INVENTION

The present inventors have now made the surprising finding that the transcription factor hypoxia induced factor (HIF)-1α is needed for normal β-cell function, that is, glucose stimulated insulin secretion. In addition, the present inventors have also shown that inducing HIF-1α in islet cells prior to transplantation improved graft survival.

Accordingly, the present invention provides a method for treating a subject having or at risk of a diabetes-related disorder, the method comprising increasing the level or stability of HIF-1α activity in pancreatic β-cells or insulin-sensitive tissues in the subject.

In one embodiment of the invention, the diabetes-related disorder is selected from the group consisting of insulitis, type 1 diabetes, type 2 diabetes, impaired glucose tolerance, gestational diabetes, insulin resistance and n-cell dysfunction.

In a further preferred embodiment of the invention, the level or stability of HIF-1α activity is increased by administering to the subject an inhibitor of a protein that mediates degradation of HIF-1α.

In one embodiment of the invention, the protein that mediates degradation of HIF-1α is a Von Hippel-Lindau protein (VHL).

In a further preferred embodiment, the inhibitor of a protein that mediates degradation of HIF-1α is an antisense nucleic acid, ribozyme, PNA, interfering RNA, siRNA, microRNA or antibody. Preferably, the inhibitor is a siRNA.

In a further preferred embodiment of the invention, the level or stability of HIF-1α activity is increased by administering to the subject a chelating agent. Preferably, the chelating agent is an iron chelator.

The iron chelator is preferably selected from the group consisting of desferrioxamine (DFO), ferrioxamine, trihydroxamic acid, CP94, EDTA, desferrioxamine hydroxamic acids, deferoxamine B (DFO) as the methanesulfonate salt, also known as desferrioxamine B mesylate (DFOM), desferal from Novartis (previously Ciba-Giegy), apoferritin, CDTA (trans-1,2-diaminocyclohexane-N,N,N′,N′-tetraacetic acid), and DTPA (diethylenetriamine-N,N,N′,N″,N″-penta-acetic acid) and cobaltous ions.

In a preferred embodiment, the iron chelator is desferrioxamine (DFO).

In another embodiment of the invention, the level of HIF-1α is increased by administering to the subject a HIF-1α polypeptide or an active fragment thereof, or a polynucleotide encoding HIF-1α polypeptide or an active fragment thereof.

In a one embodiment of the invention, the polynucleotide is a vector encoding a HIF-polypeptide or active fragment thereof. Preferably the vector is a viral vector.

In a further preferred embodiment of the invention, the vector is within a cell. Preferably, the cell is a pancreatic (3-cell. More preferably the cell is autologous.

In a further preferred embodiment of the invention, the HIF-1α polypeptide or active fragment thereof is administered with a pharmaceutically acceptable carrier.

The present invention also provides a method of transplanting pancreatic islet cells in a subject, the method comprising administering islet cells to a subject and increasing the level or activity of HIF-1α in the islet cells.

In one embodiment of the invention, the level or stability of HIF-1α in increased in the islet cells before transplantation.

In another embodiment of the invention, the level or stability of HIF-1α is increased in the islet cells after transplantation.

In a further preferred embodiment of the invention, the level or stability of HIF-1α activity is increased by administering to the subject an inhibitor of a protein that mediates degradation of HIF-1α.

In one embodiment of the invention, the protein that mediates degradation of HIF-1α is VHL.

In a further preferred embodiment of the invention, the inhibitor of a protein that mediates degradation of HIF-1α is an antisense nucleic acid, ribozyme, PNA, interfering RNA, siRNA, microRNA or antibody. Preferably, the inhibitor is a siRNA.

In a further preferred embodiment of the invention, the level or stability of HIF-1α activity is increased by administering to the subject an iron chelator.

The iron chelator is preferably selected from the group consisting of desferrioxamine (DFO), ferrioxamine, trihydroxamic acid, CP94, EDTA, desferrioxamine hydroxamic acids, deferoxamine B (DFO) as the methanesulfonate salt, also known as desferrioxamine B mesylate (DFOM), desferal from Novartis (previously Ciba-Giegy), apoferritin, CDTA (trans-1,2-diaminocyclohexane-N,N,N′,N′-tetraacetic acid), and DTPA (diethylenetriamine-N,N,N′,N″,N″-penta-acetic acid) and cobaltous ions.

In a preferred embodiment, the iron chelator is desferrioxamine (DFO).

In another embodiment of the invention the level of HIF-1α is increased by administering to the subject a HIF-1α polypeptide or an active fragment thereof, or a polynucleotide encoding HIF-1α polypeptide or an active fragment thereof.

In a one embodiment of the invention, the polynucleotide is a vector encoding a HIF-1α polypeptide or active fragment thereof. Preferably the vector is a viral vector.

In a further preferred embodiment of the invention, the vector is within a cell. Preferably, the cell is a pancreatic β-cell. More preferably the cell is autologous.

In a further preferred embodiment of the invention, the HIF-1α polypeptide or active fragment thereof is administered with a pharmaceutically acceptable carrier.

The present invention also provides a method for the treatment of a diabetes-related disorder, which involves the method of transplantation according to the methods of the invention. Preferably, the diabetes-related disorder is type 1 diabetes.

The methods of the invention can be performed on a range of different subjects. Preferably, the subject is a mammal. More preferably, the subject is human.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As will be apparent, preferred features and characteristics of one aspect of the invention are applicable to other aspects of the invention.

The invention is hereinafter described by way of the following non-limiting Examples and with reference to the accompanying figures.

### BRIEF DESCRIPTION OF THE ACCOMPANYING DRAWINGS

FIG. 1: HIF-1α is Present in Human and Mouse Islets and in Min6 Cells, and is Decreased in Islets from People with Diabetes.

(A) By real-time PCR, expression of ARNT and HIF-1α were decreased by 90% in islets from people with type 2 diabetes (white bars) compared to control subjects (black bars). Expression of AhR was also decreased. (B) In islets isolated from mice with (3-cell specific knockout of ARNT, HIF-1α expression was significantly decreased. (C) Following ARNT affinity-purification, a band corresponding to HIF-1α was present in Min6 cells basally and following treatment with DFO. (D) ARNT co-immunoprecipitated with HIF-1α, HIF-2α and AhR in Min6 cells. (E) HIF-1α co-immunoprecipitated with ARNT, both in the basal state and following treatment with DFO. (F) HIF-2α also co-immunoprecipitated with ARNT. (G) AhR did not co-immunoprecipitate with ARNT.

FIG. 2: HIF-1α Protein is Present in a Range of Normal Tissues, and in Min6 Cells.

Tissues were isolated from wild-type mice and immediately snap-frozen in liquid nitrogen. HIF-1α protein was detectable following immunoprecipitation in liver, muscle, kidney, pancreas and in Min6 cells, used as the positive control.

FIG. 3: Knockdown of HIF-1α Protein Markedly Impairs Glucose Stimulated ATP Generation, Glucose Stimulated Insulin Secretion and Gene Expression in Min6 Cells.

(A) RNAi treatment for 48 hours produced significant decreases in expression of HIF-1α, HIF-2α and AhR (p<0.01, 0.01 and 0.05 respectively). (B) In control cells, increasing glucose concentration from 1 mM to 25 mM caused a significant increase in cellular ATP concentration (p<0.05) but this was completely blocked in cells treated with RNAi directed against HIF-1α. (C) Min6 cells treated with scrambled control RNAi sequences have normal glucose stimulated insulin secretion (GSIS). Treatment with HIF-1α RNAi severely impaired GSIS, similar to that seen with ARNT RNAi. Addition of HIF-2α or HIF-2α+AhR RNAis did not cause significant further decreases in GSIS. HIF-2α RNAi alone or AhR RNAi alone produced ˜25% impairment in GSIS (data not shown). (D) HIF-1α RNAi caused significant decreases in gene expression in Min6 cells with decreased expression of the MODY genes HNF1α, PDX-1 and glucokinase (GK) and a trend to decreased HNF4α. (E) Expression of IRS-2 was significantly decreased by HIF-1α RNAi and (F) Expression of GLUT1, G6PI, aldolase (Aldo) and phosphofructokinase (PFK) mRNAs were all significantly decreased by HIF-1α RNAi.

FIG. 4: Glucose Tolerance is Abnormal in β-HIF-1α Mice.

(A) Female β-HIF-1α mice have marked glucose intolerance following intraperitoneal glucose tolerance testing (2 g/kg). (B) Male β-HIF-1α mice also have significantly worse glucose intolerance. The glucose intolerance due to a β-cell defect, demonstrated by severely impaired GSIS in both female (C) and male (D) β-HIF-1α mice. (E) Total insulin content was unchanged in islets isolated from β-HIF-1α mice compared to their controls. Despite this, the islets in vitro show marked impairment in GSIS, shown in (F).

FIG. 5: Islets from β-HIF-1α Mice were Unable to Control Glucose Post-Transplantation.

Islets were isolated from mice and transplanted into diabetic SCID mice in a 1 donor: 1 recipient ratio. Despite similar numbers of islets being isolated from β-HIF-1α and control mice, and identical total insulin content, islets from β-HIF-1αmice were unable to control glucose.

FIG. 6: Increasing HIF-1α Protein by Treatment with DFO at the Concentrations Shown for 4 Hours Markedly Improves β-Cell Function.

(A) DFO treatment did not alter expression of the house-keeping genes TATA-box binding protein (TBP) or transthyretin, or (B) increase expression of ARNT. However, at the highest dose, mRNA for HIF-1α showed increased expression. (C) DFO treatment significantly increased expression of several genes known to be important for normal β-cell function including HNF4α, GLUT1, GLUT2, phosphoglucomutase (PGM), IRS-2 and Akt2 (D) Human islets cells treated with DFO for 4 hours showed markedly improved GSIS. (E) In accord with the increased insulin release, DFO treatment was associated with increased ATP concentrations (E). Black lines indicate which columns/groups are being compared for the significance levels.

FIG. 7: DFO Treatment Improves Outcome of Minimal-Mass Human Islet Transplantation in Mice.

(A) Human islets from 3 different donors were transplanted into diabetic SCID mice either in an excess number (2000 IEQ per mouse), or in a minimal mass number (600 IEQ per mouse). The minimal mass islets were treated with control or DFO at 125 μmol/L for 2 hours prior to transplantation. As shown, large numbers of islets from each preparation were able to reverse diabetes in all recipients (n=3). As expected, 600 IEQ isolated from the same people cured diabetes in 0% (n=6). In contrast, 2 hours of culture with DFO improved outcome following transplantation of 600 IEQ to 75% (n=8). (B) Model of HIF-1α function in β-cells. DFO treatment increases HIF-1α by inhibiting its degradation, and has marked beneficial effects on β-cell function. Under normoxic conditions, HIF-1α is required for normal O-cell function, as loss by RNAi in cell culture or deletion in mice impairs GSIS. The pancreas is normally exposed to relative hypoxia (5-8% O2), and under these conditions, HIF-1α is particularly important for survival and maintenance of β-cell function. In the setting of anoxia, HIF-1α is not able to prevent β-cell demise.

### Key to Sequence Listing

SEQ ID NO: 1=Homo sapiens HIF-1α protein isoform 1 [accession no. NP_{—}001521]
SEQ ID NO: 2=Homo sapiens HIF-1α protein isoform 2 [accession no. NP_{—}851397]
SEQ ID NO: 3=Mus musculus HIF-1α protein [accession no. NP_{—}034561]
SEQ ID NO: 4=Rattus norvegicus HIF-1α protein [accession no. NP_{—}077335]
SEQ ID NO: 5=Homo sapiens HIF-1α cDNA variant 1 [accession no. NM_{—}001530]
SEQ ID NO: 6=Homo sapiens HIF-1α cDNA variant 2 [accession no. NM_{—}181054]
SEQ ID NO: 7=Mus musculus HIF-1α cDNA [accession no. NM_{—}010431]
SEQ ID NO: 8=Rattus norvegicus HIF-1α cDNA [accession no. NM_{—}024359]
SEQ ID NO: 9=Homo sapiens VHL protein isoform 1 [accession no. NP_{—}000542]
SEQ ID NO: 10=Homo sapiens VHL protein isoform 2 [accession no. NP_{—}937799]
SEQ ID NO: 11=Mus musculus VHL protein [accession no. NP_{—}033533]
SEQ ID NO: 12=Rattus norvegicus VHL protein [accession no. NP_{—}434688]
SEQ ID NO: 13=Homo sapiens VHL cDNA variant 1 [accession no. NM_{—}000551]
SEQ ID NO: 14=Homo sapiens VHL cDNA variant 2 [accession no. NM_{—}198156]
SEQ ID NO: 15=Mus musculus VHL cDNA [accession no. NM_{—}009507]
SEQ ID NO: 16=Rattus norvegicus VHL cDNA [accession no. NM_{—}052801]
SEQ ID NO: 17=VHL siRNA
SEQ ID NO: 18=VHL siRNA
SEQ ID NO: 19=VHL siRNA
SEQ ID NO: 20=VHL siRNA
SEQ ID NO: 21=Identified peptide (matched amino acid sequence for HIF-1α)
SEQ ID NO: 22=Identified peptide (matched amino acid sequence for HIF-1α)
SEQ ID NO: 23=Identified peptide (matched amino acid sequence for HIF-1α)
SEQ ID NO: 24=Identified peptide (matched amino acid sequence for HIF-1α)
SEQ ID NO: 25=Identified peptide (matched amino acid sequence for HIF-2α)
SEQ ID NO: 26=Identified peptide (matched amino acid sequence for HIF-2α)

### DETAILED DESCRIPTION OF THE PREFERRED EMBODIMENTS

### General Techniques

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

### Diabetes-Related Disorders

The present invention provides a method for treating a subject having or at risk of a diabetes-related disorder.

By “treating”, it is meant to ameliorate, inhibit, lessen, reverse, or prevent a diabetes-related disorder, or to delay onset of a diabetes-related disorder.

By “diabetes-related disorder” it is meant to include diabetes and any manifested symptoms of diabetes in any mammal, such as impaired glucose tolerance, gestational diabetes, insulin resistance, β-cell dysfunction, insulitis. Human forms include type 1 and type 2 diabetes, gestational diabetes and rare monogenic forms such as the maturity onset diabetes of the young syndromes.

By “at risk of” it is meant a subject not formally diagnosed with diabetes, but demonstrating a symptom in terms of insulin or glucose level, and susceptibility to diabetes or a related condition due to family history, genetic predisposition, or obesity in the case of type 2 diabetes, or has previously had diabetes or a related condition and is subject to risk of recurrence.

### HIF-1α Polypeptides and Polynucleotides

As used herein “HIF-1” is characterised as a DNA-binding protein which binds to a region in the regulatory, preferably in the enhancer region, of a structural gene having the HIF-1 binding motif. Included among the structural genes which can be activated by HIF-1 are erythropoietin (EPO), vascular endothelial growth factor (VEGF), and glycolytic gene transcription in cells subjected to hypoxia.

HIF-1 is composed of subunits HIF-1α and an isoform of HIF-1β. In addition to having domains which allow for their mutual association in forming HIF-1, the α and β subunits of HIF-1 both contain DNA-binding domains. The α subunit is uniquely present in HIF-1, whereas the β subunit (ARNT) is a component of at least two other transcription factors.

The methods of the present invention involve increasing the level or stability of HIF-1α in pancreatic β-cells or insulin-sensitive tissues of the subject.

By “insulin-sensitive tissues” it is meant tissues that are responsive to insulin action (including the uptake of glucose) at a clinically-normal level.

In one embodiment, the methods of the invention involve administering to the subject a HIF-1α polypeptide or an active fragment thereof, or a polynucleotide encoding HIF-1α polypeptide or an active fragment thereof.

The HIF-1α polypeptide can be a substantially purified, or recombinant polypeptide. Preferably, the HIF-1α polypeptide comprises a sequence which shares at least 75% identity with a sequence as shown in any one of SEQ ID NOS: 1 to 4.

By “substantially purified polypeptide” or “purified” we mean a polypeptide that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated.

The term “recombinant” in the context of a polypeptide refers to the polypeptide when produced by a cell, or in a cell-free expression system, in an altered amount or at an altered rate compared to its native state. In one embodiment, the cell is a cell that does not naturally produce the polypeptide. However, the cell may be a cell which comprises a non-endogenous gene that causes an altered, preferably increased, amount of the polypeptide to be produced. A recombinant polypeptide of the invention includes polypeptides which have not been separated from other components of the transgenic (recombinant) cell, or cell-free expression system, in which it is produced, and polypeptides produced in such cells or cell-free systems which are subsequently purified away from at least some other components.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogous and/or derivatives of the polypeptides of the invention as described herein.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length.

As used herein a “biologically active fragment” is a portion of a polypeptide of the invention which maintains a defined activity of the full-length polypeptide, namely be able to promote glucose stimulated insulin secretion (GSIS) and cell survival. In one embodiment, the biologically active fragment contains one and preferably both of the transactivation domains of HIF-1α. By “transactivation domains of HIF-1α” it is meant the NH₂-terminal transactivation domain (amino acids 531-575) and the COOH-terminal transactivation domain (amino acids 786-826) of HIF-1α that interact with general transcription machinery to activate transcription from promoters of HIF-1α. target genes. Biologically active fragments can be any size as long as they maintain the defined activity. Preferably, biologically active fragments are at least 100, more preferably at least 200, and even more preferably at least 350 amino acids in length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide of the invention can be subjected to in vitro mutagenesis. Such in vitro mutagenesis techniques may include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). Products derived from mutated/altered DNA can readily be screened using techniques described herein to determine if they are able to confer enhanced GSIS, and/or improved islet graft survival.

In designing amino acid sequence mutants, the location of the mutation site and the nature of the mutation will depend on characteristic(s) to be modified. The sites for mutation can be modified individually or in series, for example, by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1 under the heading of “exemplary substitutions”.

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are polypeptides of the present invention which are differentially modified during or after synthesis, for example, by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptide of the invention.

TABLE 1
Exemplary substitutions

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

In another embodiment, the methods of the invention involve administration of a polynucleotide encoding HIF-1α or an active fragment thereof. The HIF-1α polynucleotide can be an isolated or exogenous polynucleotide. Preferably, the HIF-1α polynucleotide comprises a sequence which shares at least 75% identity with a sequence as shown in any one of SEQ ID NOS: 5 to 8.

By an “isolated polynucleotide”, including DNA, RNA, or a combination of these, single or double stranded, in the sense or antisense orientation or a combination of both, dsRNA or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which they are naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. In one embodiment, the cell is a cell that does not naturally comprise the polynucleotide. However, the cell may be a cell which comprises a non-endogenous polynucleotide resulting in an altered, preferably increased, amount of production of the encoded polypeptide. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

With regard to the defined polynucleotides, it will be appreciated that % identity figures higher than those provided above will encompass preferred embodiments. Thus, where applicable, in light of the minimum % identity figures, it is preferred that a polynucleotide of the invention comprises a sequence which is at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

### Administration of HIF-1α Polypeptides and Polynucleotides

In a preferred embodiment of the invention, an HIF-1α polypeptide or active fragment thereof is administered with a biologically acceptable carrier.

The phrase, “biologically acceptable carrier” refers to any diluent, excipient, additive, or solvent which is either pharmaceutically accepted for use in the mammal for which a composition is formulated.

Routes of administration of the polypeptide or active fragment thereof include but are not limited to parenteral (for example, intravenous, intradermal, intraperitoneal or subcutaneous), oral, inhalational (for example, intranasal), transdermal (for example, topical), transmucosal, and rectal administration.

In a further preferred embodiment of the invention, the HIF-1α polynucleotide is inserted into a recombinant expression vector for the purposes of administration to the subject.

The term “recombinant expression vector” refers to a plasmid, virus or other vehicle known in the art that has been manipulated by insertion or incorporation of the HIF-1α genetic sequences. Such expression vectors contain a promoter sequence which facilitates the efficient transcription in the host of the inserted genetic sequence. The expression vector typically contains an origin of replication, a promoter, as well as specific genes which allow phenotypic selection of the transformed cells.

In one embodiment, the viral vector is derived from adeno-associated virus (AAV) and comprises a constitutive or regulatable promoter capable of driving sufficient levels of expression of the HIF-1α-encoding DNA in the viral vector. Preferably, the viral vector comprises inverted terminal repeat sequences of AAV, such as those described in WO 93/24641. In a preferred embodiment, the viral vector comprises polynucleotide sequences of the pTR-UF5 plasmid. The pTR-UF5 plasmid is a modified version of the pTR_{BS}-UF/UF1/UF2/UFB series of plasmids (Zolotukiin et al., 1996; Klein et al., 1998).

Promoters useful with the subject invention include, for example, the cytomegalovirus immediate early promoter (CMV), the human elongation factor 1-α promoter (EF1), the small nuclear RNA promoters (U1a and U1b), α-myosin heavy chain promoter, Simian virus 40 promoter (SV40), Rous sarcoma virus promoter (RSV), Adenovirus major late promoter, β-actin promoter and hybrid regulatory element comprising a CMV enhancer/β-actin promoter. These promoters have been shown to be active in a wide range of mammalian cells.

The promoters are operably linked with heterologous DNA encoding HIF-1α. By “operably linked”, it is intended that the promoter element is positioned relative to the coding sequence to be capable of effecting expression of the coding sequence.

Promoters particularly useful for expression of a protein in islet cells include, for example the insulin promoter (for example, the rat insulin promoter) and the PDX 1/IPF1 promoter.

Also contemplated for use with the vectors of the present invention are inducible and cell type specific promoters. For example, Tet-inducible promoters (Clontech, Palo Alto, Calif.) and VP16-LexA promoters (Nettelbeck et al., 1998) can be used in the present invention.

The vectors can also include introns inserted into the polynucleotide sequence of the vector as a means for increasing expression of heterologous DNA encoding HIF-1α. For example, an intron can be inserted between a promoter sequence and the region coding for the protein of interest on the vector. Introns can also be inserted in the coding regions. Transcriptional enhancer elements which can function to increase levels of transcription from a given promoter can also be included in the vectors of the invention. Enhancers can generally be placed in either orientation, 3′ or 5′, with respect to promoter sequences. In addition to the natural enhancers, synthetic enhancers can be used in the present invention. For example, a synthetic enhancer randomly assembled from Spc5-12-derived elements including muscle-specific elements, serum response factor binding element (SRE), myocyte-specific enhancer factor-1 (MEF-1), myocyte-specific enhancer factor-2 (MEF-2), transcription enhancer factor-1 (TEF-1) and SP-1 (Li et al., 1999; Deshpande et al., 1997; Stewart et al., 1996; Mitchell and Tjian, 1989; Briggs et al., 1986; Pitluk et al., 1991) can be used in vectors of the invention.

The gene therapy methods of the invention can be performed by ex vivo or in vivo treatment of the patient's cells or tissues, preferably the patient's islet cells or pancreatic tissue. The vectors of the invention can be introduced into suitable cells, cell lines or tissue using methods known in the art. The viral particles and vectors can be introduced into cells or tissue in vitro or in vivo. Methods contemplated include transfection, transduction, injection and inhalation. For example, vectors can be introduced into cells using liposomes containing the subject vectors, by direct transfection with vectors alone, electroporation or by particle bombardment. In an exemplified embodiment, islet cells are infected in vivo by injection of viral particles comprising recombinant vector into pancreatic tissue of the subject.

The dosage of recombinant vector or the virus to be administered to the subject can be determined by the ordinarily skilled clinician based on various parameters such as mode of administration, duration of treatment, the disease state or condition involved, and the like. Typically, recombinant virus of the invention is administered in doses between 10⁵ and 10¹⁴ infectious units. The recombinant vectors and virus of the present invention can be prepared in formulations using methods and materials known in the art. Numerous formulations can be found in Remington's Pharmaceutical Sciences, 15^{th} Edition (1975).

Inhibitors of Proteins that Mediate Degradation of HIF-1α

In one embodiment, the methods of the invention involve administering to the subject an inhibitor of a protein that mediates degradation of HIF-1α.

In a preferred embodiment, the protein that mediates degradation of HIF-1α is a Von Hippel-Lindau protein (VHL). Preferably, the VHL protein has a sequence which shares at least 75% identity with a sequence as shown in any one of SEQ ID NO: 9 to 12.

In a further preferred embodiment, the inhibitor of a protein that mediates degradation of HIF-1α is selected from the group consisting of an antisense polynucleotide, ribozyme, PNA, interfering RNA, siRNA, microRNA or antibody. These inhibitors are described in detail below. In a preferred embodiment, the inhibitor targets the portion of the VHL protein that binds to the oxygen degradation domain of HIF-1α.

### Antisense Polynucleotides

The term “antisense polynucleotide” shall be taken to mean a DNA or RNA, or combination thereof, molecule that is complementary to at least a portion of a specific mRNA molecule encoding a polypeptide of the invention and capable of interfering with a post-transcriptional event such as mRNA translation. The use of antisense methods is well known in the art (see for example, G. Hartmann and S. Endres, Manual of Antisense Methodology, Kluwer (1999)).

An antisense polynucleotide of the invention will hybridise to a target polynucleotide under physiological conditions. As used herein, the term “an antisense polynucleotide which hybridises under physiological conditions” means that the polynucleotide (which is fully or partially single stranded) is at least capable of forming a double stranded polynucleotide with mRNA encoding a protein, such as those encoding the VHL protein (the corresponding cDNA sequence of which is provided in any one of SEQ ID NO:13 to 16) under normal conditions in a cell, preferably a β-cell.

Antisense molecules may include sequences that correspond to the structural genes or for sequences that effect control over the gene expression or splicing event. For example, the antisense sequence may correspond to the targeted coding region of the genes of the invention, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilise the molecule.

### Catalytic Polynucleotides

The term “catalytic polynucleotide/nucleic acid” refers to a DNA molecule or DNA-containing molecule (also known in the art as a “deoxyribozyme”) or an RNA or RNA-containing molecule (also known as a “ribozyme”) which specifically recognises a distinct substrate and catalyses the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988, Perriman et al. 1992) and the hairpin ribozyme (Zolotukiin et al., 1996; Klein et al., 1998; Shippy et al., 1999).

The ribozymes of this invention and DNA encoding the ribozymes can be chemically synthesised using methods well known in the art. The ribozymes can also be prepared from a DNA molecule (that upon transcription, yields an RNA molecule) operably linked to an RNA polymerase promoter, for example, the promoter for T7 RNA polymerase or SP6 RNA polymerase. Accordingly, also provided by this invention is a nucleic acid molecule, that is, DNA or cDNA, coding for a catalytic polynucleotide of the invention. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilise the ribozyme and make it resistant to RNase.

As with antisense polynucleotides described herein, catalytic polynucleotides of the invention should also be capable of “hybridising” a target nucleic acid molecule (for example an mRNA encoding a VHL polypeptide (the corresponding cDNA sequences of which is provided in any one of SEQ ID NO: 13 to 16): under “physiological conditions”, namely those conditions within a cell (especially conditions in a β-cell).

### RNA Interference

RNA interference (RNAi) is particularly useful for specifically inhibiting the production of a particular protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a protein that mediates degradation of HIF-1α. Conveniently, the dsRNA can be produced from a single promoter in a recombinant vector or host cell, where the sense and anti-sense sequences are flanked by an unrelated sequence which enables the sense and anti-sense sequences to hybridise to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology to the target gene to be inactivated. The DNA therefore comprises both sense and antisense sequences that, when transcribed into RNA, can hybridise to form the double-stranded RNA region. In a preferred embodiment, the sense and antisense sequences are separated by a spacer region that comprises an intron which, when transcribed into RNA, is spliced out. This arrangement has been shown to result in a higher efficiency of gene silencing. The double-stranded region may comprise one or two RNA molecules, transcribed from either one DNA region or two. The presence of the double stranded molecule is thought to trigger a response from an endogenous mammalian system that destroys both the double stranded RNA and also the homologous RNA transcript from the target mammalian gene, efficiently reducing or eliminating the activity of the target gene.

The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The RNA molecule may of course comprise unrelated sequences which may function to stabilise the molecule. The RNA molecule may be expressed under the control of a RNA polymerase II or RNA polymerase III promoter. Examples of the latter include tRNA or snRNA promoters.

Preferred small interfering RNA (‘siRNA”) molecules comprise a nucleotide sequence that is identical to about 19-21 contiguous nucleotides of the target mRNA. Preferably, the siRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the mammal in which it is to be introduced, for example as determined by standard BLAST search. Examples of siRNA molecules that target VHL mRNA are provided in any one of SEQ ID NO:17 to 20.

microRNA

MicroRNA regulation is a clearly specialised branch of the RNA silencing pathway that evolved towards gene regulation, diverging from conventional RNAi/PTGS. MicroRNAs are a specific class of small RNAs that are encoded in gene-like elements organised in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al. 2005; Almeida and Allshire, 2005).

### Polyclonal and Monoclonal Antibodies

If polyclonal antibodies are desired, a selected mammal (for example, mouse, rabbit, goat, horse, etc.) is immunised with an immunogenic polypeptide such as VHL (for example, as shown in any one of SEQ ID NO:9 to 12). Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides peptides of the invention or fragments thereof haptenised to another peptide for use as immunogens in animals.

Monoclonal antibodies directed against a protein that mediates the degradation of HIF-1 cc can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; that is, for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

### Chelating Agents

In one preferred embodiment of the invention, the level or stability of HIF-1α activity is increased by administering to the subject a chelating agent.

A “chelating agent” refers to a substance, compound, mixture, or formulation capable of having an affinity for iron, copper or other transition metal and which is capable of binding iron or copper or any other transition metal in vitro or in vivo. When used in this invention, the chelating agent is useful in chelating/binding ferrous iron or copper or other transition metal and/or decreasing oxidative stress by acting as a transition metal sequestrant and/or antioxidant.

In a preferred embodiment, the chelating agent is an iron chelator.

The iron chelator is preferably selected from the group consisting of desferrioxamine (DFO), ferrioxamine, trihydroxamic acid, CP94, EDTA, desferrioxamine hydroxamic acids, deferoxamine B (DFO) as the methanesulfonate salt, also known as desferrioxamine B mesylate (DFOM), desferal from Novartis (previously Ciba-Giegy), apoferritin, CDTA (trans-1,2-diaminocyclohexane-N,N,N′,N′-tetraacetic acid), and DTPA (diethylenetriamine-N,N,N′,N″,N″-penta-acetic acid) and cobaltous ions.

The chelating agent may be administered by any suitable route. Routes of administration of the chelating agent include intramuscular, parenteral (including intravenous), intraarterial, subcutaneous, oral, and nasal administration.

Preferably, the chelating agent is administered in at least one dose that is within the range 0.0001 to 1.0 mg·kg.

In a preferred embodiment, the iron chelator is desferrioxamine (DFO).

In a further preferred embodiment the DFO is administered intravenously, diluted in normal saline. Preferably, the dose is within the range 5 g to 10 g per person. Preferably the dose is administered once weekly.

### EXAMPLES

### Example 1

### Materials and Methods

### Human Pancreatic Islets

Human pancreatic islets were purified from seven normoglycemic subjects using the modified Ricordi method (Ricordi et al., 1988) as previously described (Gunton et al., 2005). The subjects are described in (Gunton et al., 2005), and were matched for age and body-mass-index. Gene expression was measured by real-time-PCR and was performed in a two-step reaction using the Invitrogen RT-for-PCR kit. The second step was performed in a fluorescent temperature cycler (ABI-Prism 7700 Sequence Detection System, Applied Biosystems) with LightCycler-RNA Master SYBR-Green-I (Roche, Mannheim, Germany) and specific primers for each of the genes (sequences available on request). Every plate included a control gene (TATA-box binding protein/TBP) for every subject. Results were analysed by unpaired t-test.

Islet Isolation from Mice

Pancreatic islets were isolated from mice aged 9-12 weeks, as previously described (Kulkarni et al., 1999; Gunton et al., 2005).

### Affinity Purification, Mass Spectrometry and Co-immunoprecipitation

ARNT2, HIF1α and HIF2α/EPAS1 antibodies were purchased from Novus Biologicals (Littleton, Colo.), AhR antibodies from Orbigen (San Diego, Calif.) and ARNT antibodies from BD Biosciences. Anti-mouse and anti-rabbit secondary antibodies were purchased from Santa Cruz (Santa Cruz, Calif.).

ARNT-affinity-purification was done by binding ARNT antibody (12 μg) to 1 ml of packed protein A/G beads in 5 ml columns. No-antibody columns were used for control samples. Columns were washed with 20 mls of PBST to remove unbound antibody. Min6 cells were grown to 80-90% confluence in 4 20 cm dishes per condition. They were washed twice in PBS and placed in serum free DMEM at 25 mM glucose for 4 hours. DFO treatment was applied at 125 μM for 4 hours to the appropriate plates, or an equal volume of vehicle to control plates. Cells were collected by scraping into LID cell lysis buffer with protease and phosphatase inhibitors as previously described (Gunton et al., 2003). After centrifugation, nuclei were disrupted by sonication. The cytoplasmic or nuclear extracts were applied to the columns as indicated, and the flow-through re-applied twice to obtain maximal binding. After this, the columns were washed twice with 20 mls of LID buffer followed by 2 washes with 20 mls of PBST. The bound proteins were eluted with reducing sample buffer.

The eluted proteins were size-separated by 10% SDS-PAGE followed by staining for proteins with Coomassie blue.

For mass spectrometry, gel slices were digested with 5 ng/ml sequencing grade modified trypsin (Promega, Madison, Wis.) in 25 mM ammonium bicarbonate containing 0.01% n-octylglucoside for 18 hrs at 37° C. Peptides were eluted from the gel slices with 80% acetonitrile, 1% formic acid. Tryptic digests were separated by capillary HPLC (C18, 75 mM i.d. Picofrit column, New Objective, Woburn, Mass.) using a flow rate of 100 nl/min over a 3 hour reverse phase gradient and analysed using a LTQ linear Ion Trap LC/MSⁿ system (Thermo Electron, San Jose, Calif.). Resultant MS/MS spectra were searched against the NCBI nr database using TurboSequest (BioWorks 3.1, Thermo Electron) with cross-correlation scores>1.5, 2.0 and 2.5 for charge states U′, u and [symbol] , respectively, >30% fragment ions, and Rsp<3. Proteins were identified with >2 unique peptide matches.

Co-immunoprecipitation studies were performed using 2 μg of the indicated antibody, overnight incubation with the indicated cell-lysate, washing, elution with reducing sample buffer, and separation by 10% SDS-PAGE. Proteins were detected with the indicated antibody followed by the appropriate HRP-conjugated secondary antibody and detection by enhanced chemiluminescence.

### Measurement of Intracellular ATP Concentrations

ATP concentrations were measured in islets and in Min6 cells following exposure to low (1 mM) or high glucose (25 mM) for 30 minutes. At the 30 minute timepoint, cells were placed on ice, washed twice in ice-cold PBS and lysed and ATP was measured using a kit purchased from Roche, according to the manufacturer's instructions.

### Small Interfering RNA (RNAi) Treatment of Min6 Cells and Insulin Release

Using Min6 cells, HIF-1α was decreased by 48 hours of treatment with small interfering RNA (siRNA/RNAi) “smartpool” (Dharmacon, Lafayette, Colo.), transfected using Lipofectamine 2000 (Invitrogen), according to the respective manufacturers' protocols. Scrambled-sequence RNAi was used as a control in all experiments.

Glucose-stimulated insulin secretion (GSIS) was assessed in triplicate wells in 3 separate experiments, and corrected for total insulin content, which did not change in treated cells (data not shown).

In separate experiments, treated cells were lysed, and RNA isolated for real-time-PCR.

### Generation of β-Cell-Specific HIF-1α Knockout Mice

β-cell-specific HIF-1α knockout mice β-HIF-1α) were generated using the Cre-lox system. Mice with floxed HIF-1α as previously described (Tomita et al., 2003) were bred with mice expressing Cre under control of the Rat Insulin Promoter (RIP-Cre mice). The RIP-Cre-alone mice have normal glucose tolerance (data not shown).

Transplantation of Islets Isolated from β-HIF-1α Knockout Mice or Floxed-Controls

Islets were isolated from mice as described above. Islets were transplanted into immunodeficient mice (SCID) which were rendered diabetic by injection of 80 mg/kg of Alloxan IV. Blood glucose was monitored in the recipient mice by tail-nick 3 times a week. At 28 days, nephrectomy was performed to exclude regeneration of endogenous β-cells in the recipient mice. The kidney was collected for fresh-frozen section as described above for pancreatic sections and the graft was examined following H&E staining.

### Example 2

### Expression of Hypoxia-Inducible Factor-1α (HIF-1α) is Decreased in Islets Isolated from People with Type 2 Diabetes

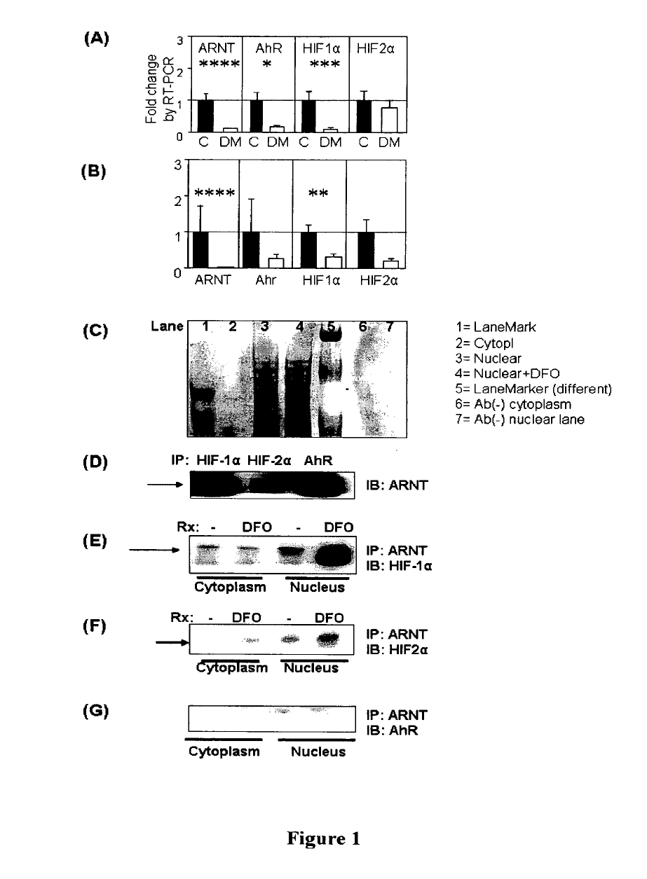

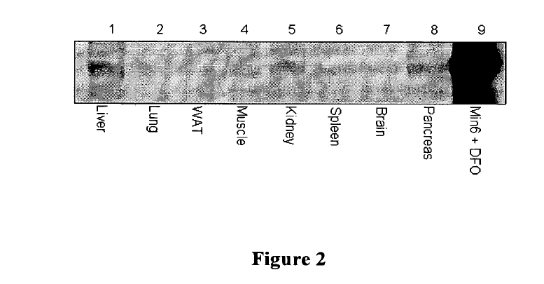

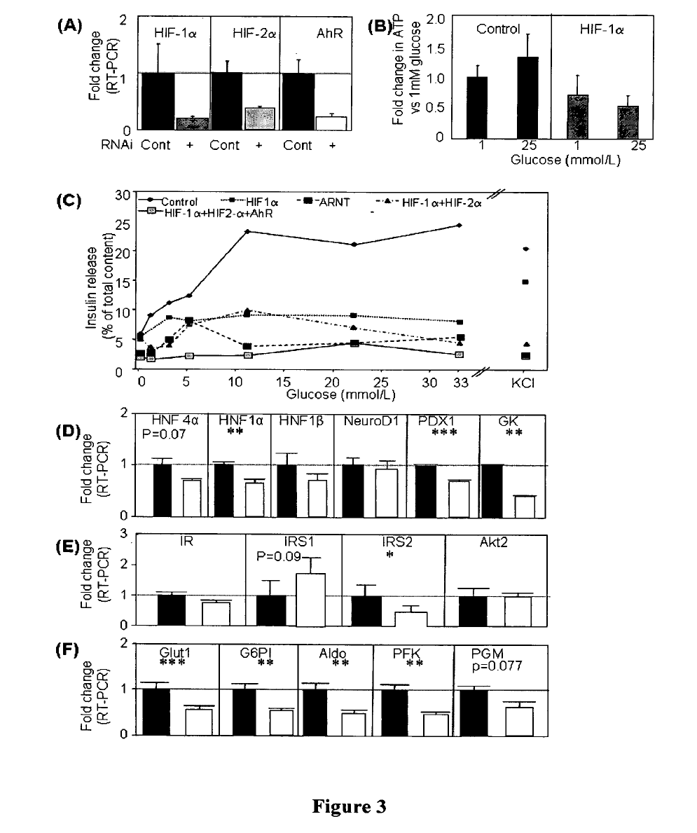

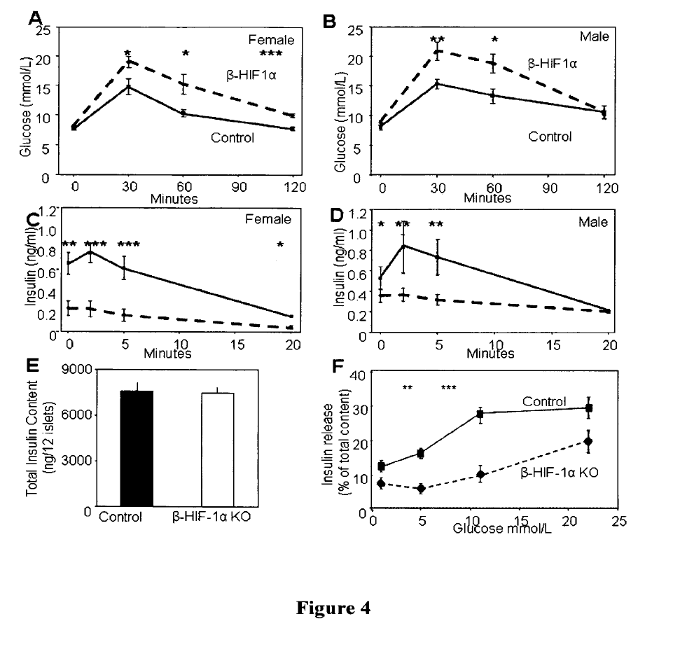

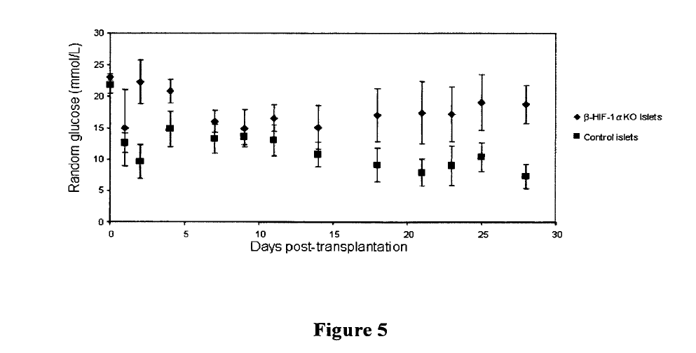

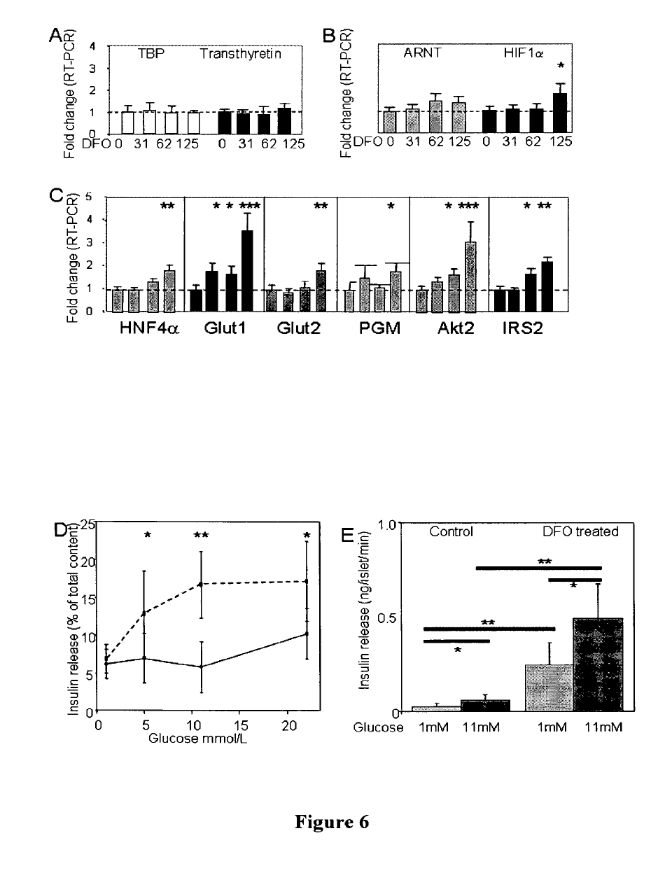

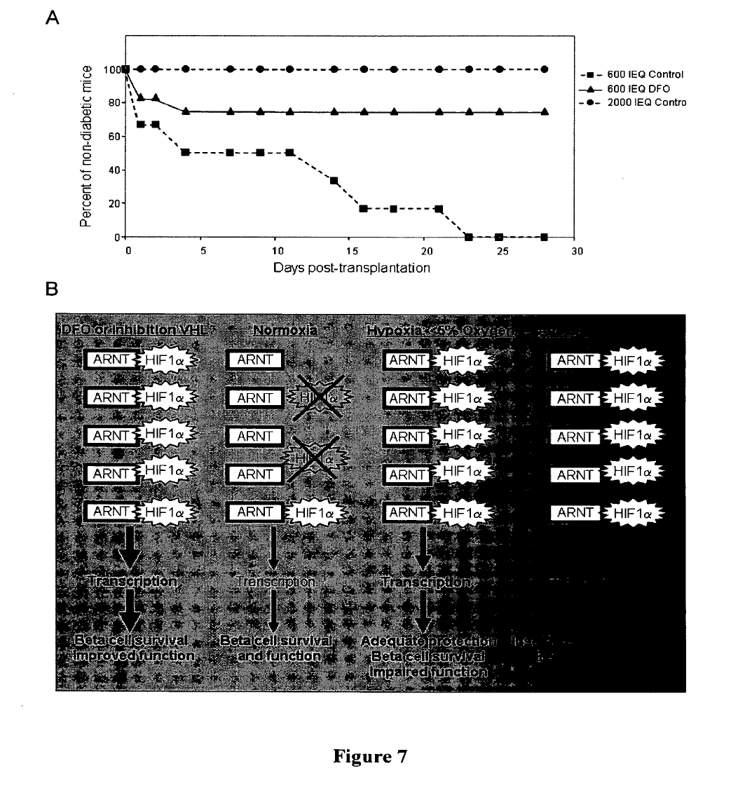

The present inventors measured HIF-1α gene expression in human islets isolated from people with normal glucose tolerance or type 2 diabetes. The islet donors have been previously described (Gunton et al., 2005). The present inventors also measured expression of other bHLH-PAS family members, aryl hydrocarbon receptor (AhR) and HIF-2α. By real-time PCR, expression of HIF-1α, HIF-2α/EPAS 1 and AhR were all clearly detected in human islets (FIG. 1A), as was ARNT. HIF-1α mRNA expression was decreased by 90% in islets from people with type 2 diabetes (FIG. 1A, p<0.001). As previously reported by the inventors (Gunton et al., 2005), there was also a 90% decrease in expression of ARNT. Expression of AhR was also significantly decreased (p<0.05) and there was no significant change in expression of HIF-2α.

### Example 3

### HIF-1α Expression is Decreased in Islets from β-Cell Specific ARNT Knockout Mice

In isolated murine islets, expression of HIF-1α, HIF-2α, AhR and ARNT mRNAs were clearly detected. Interestingly, in β-cell specific ARNT knockout mice (white bars), as in type 2 diabetes islets which had decreased ARNT, HIF-1α expression was markedly decreased (FIG. 1B), suggesting a potential role for ARNT in the transcriptional regulation of HIF-1α at the mRNA level.

### Example 4

### HIF-1α has a Direct Protein-Protein Interaction with ARNT in Min6 Cells in the Basal State and Following Exposure to Hypoxia-Mimics

To determine which bHLH-PAS transcription factors were bound to ARNT and thus were potentially transcriptionally active, the inventors used ARNT-affinity purification and mass spectrometry as described in materials and methods. From this, the inventors identified peptides which matched the amino acid sequences for HIF-1α (SIYEYYHALDSDHLTK (SEQ ID NO: 21), PPM*TCLVLICEPIPHPSNIEIPLDSK (SEQ ID NO: 22), TFLSRHSLDMK#FSYCDER (SEQ ID NO: 23) and TM*NIKSATWK (SEQ ID NO: 24)), and HIF-2α (ENLTLK#NGSGFGK (SEQ ID NO: 25) and M*RSAKDFGAR (SEQ ID NO: 26)) in the nuclear fractions of Min6 cells.

Of importance, the HIF-1α peptides were identified in both the basal state (Lane 3, FIG. 1C) and following treatment with the hypoxia-mimic desferrioxamine (DFO) (Lane 4, FIG. 1C), whereas HIF-2α was only identified following DFO treatment (Lane 4, FIG. 1C). This suggested that HIF-1α protein may escape degradation in Min6 cells without hypoxia or cytokine treatment, that is, in the basal state.

The inventors confirmed that HIF-1α was bound to ARNT in both the basal state and following DFO treatment by co-immunoprecipitation studies. Antibodies to HIF-1α, HIF-2α and AhR were able to precipitate ARNT from the Min6 lysates as shown in FIG. 1D. Reciprocally, antibodies to ARNT co-immunoprecipitated HIF-1α from the nuclear fraction of Min6 cells both in the basal state (Lane 3, FIG. 1E) and following stimulation of HIF-1α protein by treatment with DFO (Lane 4, FIG. 1E). In an analogous fashion, FIG. 1F shows that anti-ARNT antibodies also co-precipitated HIF-2α in the basal (Lane 3) and DFO-stimulated states (Lane 4), although with lower efficacy. In contrast, FIG. 1G shows that anti-ARNT antibodies did not co-precipitate detectable amounts of Ahr in Min6 cell lysates from either the nuclear or the cytoplasmic components, despite clearly detecting AhR protein by Western blot (data not shown).

### Example 5

### HIF-1α Protein is Detectable in Pancreas and Islets in the Basal State, And HIF-1α Protein is Decreased in the Islets of People with Type 2 Diabetes

Since HIF-1α is tightly regulated at the protein level, the present inventors sought to investigate whether the HIF-1α protein was present in normal pancreas. Immunoprecipitation studies were performed from a range of mouse tissues as shown in FIG. 2. As shown in Lane 8, whole pancreas has readily detectable amounts of HIF-1α protein. Muscle has been previously reported to express significant amounts of HIF-1α protein, and this experiment reproduces that finding (Lane 4).

### Example 6

### HIF-1α Knockdown by RNA-Interference Severely Impairs Glucose-Stimulated Insulin Release in Min6 Cells

To clarify the function/roles of HIF-1α, HIF-2α and AhR in β-cells, the present inventors used the murine insulinoma-derived (3-cell line of Min6 cells, which maintain GSIS. Using RNA interference (RNAi), the present inventors knocked down expression of AhR, HIF-1α and HIF-2α individually and in combination. As shown in FIG. 3A, HIF-1α and AhR RNAi both achieved ˜75% knockdown, and HIF-2α RNAi caused ˜65% knockdown.

In FIG. 3B, the present inventors show that in control RNAi treated Min6 cells, intracellular ATP concentration increases by 40% following exposure to 25 mM glucose. However, in cells treated with HIF-1α RNAi, there was no increase in intracellular ATP. Consistent with these findings, FIG. 3C shows that decreasing HIF-1α caused a severe impairment in GSIS. Interestingly, HIF-1α RNAi did not impair KCl stimulated insulin release (data not shown), suggesting a specific glucose-sensing defect. Combining HIF-1α with HIF-2α RNAi to decrease expression of both genes did not further impair GSIS, but combination of HIF-1α, HIF-2α and AhR produced a defect in GSIS which did not differ significantly from that produced by ARNT RNAi, suggesting a small but significant role of AhR in n-cell function. This is consistent with the fact that 23% of AhR knockout mice develop glucose intolerance by 8 months of age (Fernandez-Salguero et al., 1997).

### Example 7

### HIF-1α RNA-Interference Impairs Gene Expression

Following treatment with HIF-1α RNAi, the present inventors measured expression of genes in the maturity onset diabetes of the young (MODY) family, insulin-signalling and glucose-uptake and glycolytic genes by real-time PCR. The present inventors found substantially decreased expression of 3 of the MODY genes: TCF1 (encoding hepatocyte nuclear factor (HNF)-1α), PDX1/IPF-1 and glucokinase (GK) (FIG. 3D). HNF4α also showed a trend towards decreased expression following HIF-1αknockdown (p<0.07). In the insulin signaling pathway, HIF-1α knockdown decreased insulin-receptor substrate (IRS)-2 mRNA (FIG. 3E). HIF-1α has been reported to regulate expression of glycolytic genes in other tissues, and consistent with this, FIG. 3F shows that decreasing HIF-1α caused markedly decreased expression of the glucose transporters GLUT1 and GLUT2 (data not shown), and several components of the glycolytic pathway including glucose-6-phosphoisomerase (G6PI), phosphofructokinase (PFK), aldolase and phosphoglucomutase (PGM) (all p<0.01).

### Example 8

### β-Cell Specific HIF-1α Knockout Mice (β-HIF-1α) are Glucose Intolerant With Failure of Glucose Stimulated Insulin Secretion

Whole-body deletion of HIF-1α is embryonic lethal in mice (Hofer et al., 2002; Iyer et al., 1998; Kotch et al., 1999), so in order to study the role of HIF-1α in β-cell function in vivo, the present inventors generated β-cell HIF-1α knockout mice using the Cre-lox system, with Cre under control of the rat insulin promoter (RIP-Cre). The RIP-Cre mice have normal glucose tolerance and insulin release (data not shown). The mice were born in a normal Mendelian distribution, were of normal weight and size and were fertile.

On glucose tolerance testing (GTT), both female and male β-HIF-1α mice showed marked glucose intolerance (FIGS. 4A and 4B), both p<0.001 by ANOVA for repeated measures. By measuring GSIS, the present inventors found that the glucose intolerance is caused by failure of first-phase insulin secretion in both female (FIG. 4C) and male mice (FIG. 4D). The female mice display prolonged elevation in their glucose at 120 minutes into the GTT, and consistent with this, female mice also have slightly lower circulating insulin at 20 minutes into the GSIS, showing that second phase insulin release is also impaired in female mice (FIG. 4C).

### Example 9

### Islets from β-HIF-1α Mice Show a Marked Right-Shift in Glucose Stimulated Insulin Release

Islets were isolated from β-HIF-1α mice as described in Methods and Materials. FIG. 4E shows that there were no significant differences in total insulin content between knockout and floxed-control mice. However, FIG. 4F shows that islets from β-HIF-1α knockout mice had markedly impaired insulin secretion at glucose concentrations of 5 mM (p=0.016) and 11 mM (p=0.001). At 22 mM glucose, the difference was no longer statistically significant (p=0.116), suggesting a right-shift in GSIS.

### Example 10

### Islets with β-Cell Deletion of HIF-1α Show Primary-Non-Function with Failure of Glycemic Control Following Islet Transplantation

Isolated human or rat islets exposed to 1% oxygen for 24 hours show central cell death, demonstrating that islets are sensitive to hypoxia (Giuliani et al., 2005). In order to investigate the role of HIF-1α in islet transplantation with relevance to the human model, the present inventors performed minimal-mass islet transplantation of islets isolated from β-HIF-1α knockout mice or their floxed controls into SCID mice which had been rendered diabetic by 70 mg/kg of Alloxan IV.

As shown in FIG. 5, β-HIF-1α knockout islets were less able to control glucose post-transplantation in this model, with average random glucose levels of 18.7±3.0 versus 7.3±1.9 mmol/L at day 28 post-transplantation.

### Example 11

### Increased HIF-1α Protein in Min6 Cells Improves Gene Expression and Glucose Stimulated Insulin Release

Because HIF-1α has been associated with protection from apoptosis during carcinogenesis, the present investigators examined the effects of increasing HIF-1α upon β-cell function.

Min6 cells were treated with DFO at the doses indicated for 4 hours. As shown in FIG. 6A, DFO treatment did not increase expression of either of the housekeeping genes TATA-box binding protein (TBP) or transthyretin. There was a small but significant increase in HIF-1α expression at the highest dose of DFO treatment (FIG. 6B) again consistent with a possible auto-regulation of HIF-1. DFO treatment dose-dependently increased expression of mRNAs for Akt2 and GLUT1 (both p<0.001), GLUT2, HNF4α, and IRS2 (all p<0.01) and phosphoglucomutase (PGM) (p<0.05). Expression of HNF1α, HNF1β, and insulin receptor did not alter with DFO treatment (data not shown).

As expected, given the increases in expression of glucose transporters and glycolytic enzymes, DFO treatment increased ATP generation in Min6 cells (FIG. 6E).

In association with the improved gene expression and ATP generation, DFO treatment also caused a substantial increase in GSIS (FIG. 6D). The present inventors were interested to determine whether DFO was beneficial for function of human islets. Islets were isolated from people with normal glucose tolerance and treated with control media or media supplemented with DFO for 4 hours, and insulin release subsequently measured at low (5 mM) and high (11 mM) glucose concentrations. As FIG. 6D shows, insulin release was markedly increased in DFO treated samples.

### Example 12

### Induction of Hypoxia Inducible Factors with DFO but not with 5% Oxygen Pre-Treatment Significantly Improves Outcome of Islet Transplantation in Mice

Islets were isolated from control mice and cultured with either control media or control media plus 125 μM DFO under normoxic conditions or in control media under hypoxic conditions (5% O2) for 2 hours before transplantation. All the islets from 1 donor mouse were transplanted into 1 recipient mouse which had been rendered diabetic (random glucose>22 mM) by streptozotocin treatment.

Transplant recipients were followed for 28 days, at which time nephrectomy was performed to confirm recurrence of diabetes.

In control treated islets, mean glucose after transplantation was 16.4 mM. In DFO treated islets, the average glucose was 11.4 mM (p=0.01) and in hypoxic treated islets was 15.5 mM (p=0.013 versus DFO treated and p=ns versus control treatment).

### Example 13

### Treatment with DFO for 4 Hours Pre-Transplant Improves Outcome of Minimal Mass Human Islet Transplantation

Human pancreatic islets were isolated from normal glucose tolerant donors as previously described (Gunton et al., 2005). Islets were transplanted under the left kidney capsule of SCID mice which had been rendered diabetic by injection of Alloxan at 95 mg/kg intravenously. Islets were transplanted in adequate mass (2000 IEQ per mouse), or minimal mass (600 IEQ per mouse). In each of the islet isolations, 2000 control-treated IEQ per mouse was adequate to cure diabetes. As expected, 600 control-treated IEQ per mouse was not adequate to control glucose at day 28 post-transplant in any mouse. Treatment with DFO at 125 μM for 4 hours pre-transplantation increased the success rate following transplant of 600 IEQ from 0% to 75%, p<0.00001 by Chi-Square.

### REFERENCES

Almeida and Allshire (2005), TRENDS Cell Biol., 15:251-258.
Briggs et al. (1986), Science, 234:47-52.
Cetkovic et al. (1994), Cytokines, 6(4):399-406.
Deshpande et al. (1997), J. Biol. Chem., 272(16):10664-10668.
Femandez-Salguero et al. (1997) Vet. Pathol., 34:605-614.
Giuliani et al. (2005), Cell Transplant., 14:67-76.
Gunton et al. (2003), J. Clin. Endocrinol. Metab., 88:1323-1332.
Gunton et al. (2005), Cell, 122:337-349.
Harayama (1998), Trends Biotechnol., 16: 76-82.
Haseloff and Gerlach (1988), Nature, 334:585-591.
Hofer et al. (2002), Pflugers Arch, 443:503-507.
Iyer et al. (1998), Genes Dev., 12:149-162.
Klein et al. (1998), Exp. Neurol., 150:183-194.
Kotch et al. (1999), Dev. Biol., 209:254-267.
Kulkami et al. (1999), Cell, 96:329-339.
Li et al. (1999), Nat. Biotechnol., 17(3):241-245.
Mandrup-Poulsen (1996), Diabetologia, 39:1005-1029.
Millar and Waterhouse (2005), Funct. Integr. Genomics, 5:129-135.
Mitchell and Tjian (1989), Science, 245:371-378.
Needleman and Wunsch (1970), J. Mol. Biol., 48:443-453.
Nettelbeck et al. (1998), Gene Ther., 5(12)1656-1664.
Pasquinelli et al. (2005), Curr. Opin. Genet. Develop., 15:200-205.
Perriman et al. (1992), Gene, 113:157-163.
Pitluk et al. (1991), J. Virol., 65:6661-6670.
Ricordi et al. (1988), Diabetes, 37:413-420.
Shippy et al. (1999), Mol. Biotech., 12:117-129.
Smith et al. (2000), Nature, 407:319-320.
Stewart et al. (1996), Genomics, 37(1):68-76.
Tomita et al. (2003), Mol. Cell. Biol., 23(19):6739-6749.
Waterhouse et al. (1998), Proc. Natl. Acad. Sci. USA, 95:13959-13964.
Zolotukiin et al. (1996), J. Virol., 70(7):4646-4654.

1. A method for treating a subject having or at risk of a diabetes-related disorder, the method comprising increasing the level or activity of Hypoxia Induced Factor 1α (HIF-1α) in pancreatic β-cells or insulin-sensitive tissues in the subject by administering to the subject an inhibitor of a protein that decreases the level or activity of HIF-1α.

2. The method of claim 1, wherein the diabetes-related disorder is selected from the group consisting of type 1 diabetes, type 2 diabetes, impaired glucose tolerance, gestational diabetes, insulin resistance and β-cell dysfunction.

3. The method of claim 1 or claim 2, wherein the protein that decreases the level or activity of HIF-1α is a Von Hippel-Lindau protein (VHL).

4. The method of any one of claims 1 to 3, wherein the inhibitor is an agent that promotes the dissociation of HIF-1α and the protein that decreases the level or activity of HIF-1α.

5. The method of claim 4, wherein the agent is a chelating agent.

6. The method of claim 5, wherein the chelating agent is selected from the group consisting of desferrioxamine (DFO), ferrioxamine, trihydroxamic acid, CP94, EDTA, desferrioxamine hydroxamic acids, deferoxamine B (DFO) as the methanesulfonate salt, also known as desferrioxamine B mesylate (DFOM), desferal from Novartis (previously Ciba-Giegy), apoferritin, CDTA (trans-1,2-diaminocyclohexane-N,N,N′,N′-tetraacetic acid), and DTPA (diethylenetriamine-N,N,N′,N″,N″-penta-acetic acid) and cobaltous ions.

7. The method of claim 6, wherein the chelating agent is desferrioxamine (DFO).

8. The method of any one of claims 1 to 3, wherein the inhibitor is an antibody, antisense nucleic acid, ribozyme, PNA, interfering RNA or siRNA.

9. The method of claim 8, wherein the inhibitor is siRNA.

10. The method of any one of claims 1 to 9, wherein the subject is human.

11. A method of transplanting pancreatic islet cells in a subject, the method comprising:
(i) administering islet cells to the subject; and
(ii) increasing the level or activity of HIF-1α in the islet cells by administering to the subject an inhibitor of a protein that decreases the level or activity of HIF-1α.

12. The method of claim 11, wherein the level or activity of HIF-1α is increased in the islet cells before transplantation.

13. The method of claim 11, wherein the level or activity of HIF-1α is increased in the islet cells after transplantation.

14. The method of any one of claims 11 to 13, wherein the protein that decreases the level or activity of HIF-1α is a Von Hippel-Lindau protein (VHL).

15. The method of any one of claims 11 to 14, wherein the inhibitor is an agent that promotes the dissociation of HIF-1α and the protein that decreases the level or activity of HIF-1α.

16. The method of claim 15, wherein the agent is a chelating agent.

17. The method of claim 16, wherein the chelating agent is selected from the group consisting of desferrioxamine (DFO), ferrioxamine, trihydroxamic acid, CP94, EDTA, desferrioxamine hydroxamic acids, deferoxamine B (DFO) as the methanesulfonate salt, also known as desferrioxamine B mesylate (DFOM), desferal from Novartis (previously Ciba-Giegy), apoferritin, CDTA (trans-1,2-diaminocyclohexane-N,N,N′,N′-tetraacetic acid), and DTPA (diethylenetriamine-N,N,N′,N″,N″-penta-acetic acid) and cobaltous ions.

18. The method of claim 17, wherein the chelating agent is desferrioxamine (DFO).

19. The method of any one of claims 11 to 14, wherein the inhibitor is an antibody, antisense nucleic acid, ribozyme, PNA, interfering RNA or siRNA.

20. The method of claim 19, wherein the inhibitor is siRNA.

21. The method of any one of claims 11 to 20, wherein the subject is human.

22. A method for the treatment of a diabetes-related disorder, which involves the method of transplantation according to any one of claims 11 to 21.

23. The method of claim 22, wherein the diabetes-related disorder is type 1 diabetes.
